(19) United States
(12) Patent Application Publication
(10) Pub. No.: US 2025/0312591 A1
(43) Pub. Date: Oct. 9, 2025

(54) ELECTROTHERAPY SYSTEM AND APPLICATIONS OF SAME

(71) Applicant: NORTHWESTERN UNIVERSITY, Evanston, IL (US)

(72) Inventors: Joseph Woojin Song, Evanston, IL (US); John A. Rogers, Wilmette, IL (US); Guillermo A. Ameer, Evanston, IL (US); Hanjun Ryu, Anseong-si, Gyeonggi-do (KR)

(21) Appl. No.: 18/865,772

(22) PCT Filed: May 18, 2023
(86) PCT No.: PCT/US2023/022651

§ 371 Date: Nov. 14, 2024

Related U.S. Application Data
(60) Provisional application No. 63/343,417, filed on May 18, 2022.

(51) Int. Cl. A61N 1/04; H02J 50/00

(52) U.S. Cl. CPC A61N 1/0468; A61N 1/0476; H02J 50/001

(57) ABSTRACT

The invention relates to an electrotherapy system comprising a pair of electrodes coupled with a region of interest of a subject for providing electrostimulation thereto; and a wireless platform coupled with the pair of electrodes for operable providing power to the stimulator.

### CROSS-REFERENCE TO RELATED PATENT APPLICATIONS

This application claims priority to and the benefit of U.S. Provisional Application No. 63/343,417, filed May 18, 2022, which is incorporated herein in its entirety by reference.

### STATEMENT AS TO RIGHTS UNDER FEDERALLY-SPONSORED RESEARCH

This invention was made with government support under grant number DK131302 awarded by the National Institutes of Health. The government has certain rights in the invention.

### FIELD OF THE INVENTION

The invention relates generally to the field of electrotherapy, and more particularly to miniaturized, bioresorbable, wireless, and battery-free electrotherapy systems and applications of the same.

### BACKGROUND OF THE INVENTION

The background description provided herein is for the purpose of generally presenting the context of the invention. The subject matter discussed in the background of the invention section should not be assumed to be prior art merely as a result of its mention in the background of the invention section. Similarly, a problem mentioned in the background of the invention section or associated with the subject matter of the background of the invention section should not be assumed to have been previously recognized in the prior art. The subject matter in the background of the invention section merely represents different approaches, which in and of themselves may also be inventions. Work of the presently named inventors, to the extent it is described in the background of the invention section, as well as aspects of the description that may not otherwise qualify as prior art at the time of filing, are neither expressly nor impliedly admitted as prior art against the invention.

Diabetes mellitus is a major public health problem that imposes significant productivity and financial burdens on society, with healthcare costs in the US exceeding $327 billion annually and projected to increase at a rate of one billion per year and contributing to the increase in years lived with disability. One of the severe complications of the approximately 30 million people living with diabetes in the United States is diabetic foot ulcers (DFUs), which occur in 15-25 percent of patients with diabetes. If not appropriately treated, these and other types of chronic wounds may lead to amputations. In fact, diabetic-related complications with chronic wounds such as DFUs are the number one cause of non-traumatic lower limb amputations worldwide. Although wound care management is well established, the multifactorial etiology, patient-specific circumstances, high regulatory and market barriers to entry, adoption for new biologics-based therapies, and adequate access to care remain challenges to effective treatment. Therefore, research into new strategies and associated technologies that prevent or improve the outcome of chronic DFUs must be developed.

A variety of strategies have been investigated to address the problems that contribute to chronic DFUs, such as impaired angiogenesis, reduced dermal cell migration and proliferation, excessive oxidative stress, prolonged inflammation, and bacterial infection. Methods include the release of drugs and biologics at the wound, the use of bioactive materials as dressings, cell transplantation, tissue-engineered or skin equivalent products, the use of vacuum, and electrotherapy. Many of the experimental approaches show promise in preclinical models and some clinical trials; nevertheless, they face significant regulatory, manufacturing, user adoption hurdles, and high development costs. Products that are in clinical use can be too expensive for widespread application, as in the case of biologics, and/or they do not fully address the underlying problems that contribute to chronic wounds. An additional unmet need is in capabilities to monitor the status of the wound to better inform clinical decisions and improve the effectiveness of therapies.

Electrotherapy has been used and investigated as a method to accelerate the closure of skin wounds. Related electrical approaches may also enable simultaneous monitoring of wound status. The hypothesis is that applied electric fields restore endogenous wound currents, to recapitulate the natural healing mechanism. Although case studies suggest that electrostimulation is effective in wound closure, its use is not widespread in clinical practice. Reasons for this limited adoption include lack of understanding of the optimal settings (e.g., for dosing, timing, and type of electrical stimulation), inadequate form factors in the hardware (e.g., use of bulky equipment that requires inpatient care and leads to decreased patient compliance), and poor control interfaces with cumbersome modes of use (e.g., the treatment often must be applied daily). A dominating additional concern for any type of therapeutic or diagnostic device that requires direct physical interfaces with the wound site is in the potential for damage to fragile soft tissues during removal after a period of use.

Therefore, a heretofore unaddressed need exists in the art to address the aforementioned deficiencies and inadequacies.

### SUMMARY OF THE INVENTION

In light of the foregoing, one aspect of this invention discloses an electrotherapy system, comprising a pair of electrodes coupled with a region of interest of a subject for providing electrostimulation thereto; and a wireless platform coupled with the pair of electrodes for operable providing power to the stimulator.

In one embodiment, the pair of electrodes has a first electrode attached onto the region of interest and a second electrode surrounding the first electrode.

In one embodiment, the pair of electrodes is spatially apart from each other to define an electrode spacing that is larger than 1 mm.

In one embodiment, the first electrode is an inner electrode placed at the center of the region of interest and the second electrode is an outer electrode placed slightly outside of the region of interest around its perimeter.

In one embodiment, the first electrode and the second electrode are concentrically arranged such that the pair of electrodes is a concentric pair of electrodes.

In one embodiment, each of the pair of electrodes is formed with a filamentary serpentine layout so that each electrode is mechanical flexible and stretchable.

In one embodiment, each electrode has a thickness in a range of 10-30 μm and a width in range of 50-200 μm.

In one embodiment, the first electrode includes a serpentine trace with a thickness of 15 μm and width of 120 μm in a flower-like design; and the second electrode adopts a similar serpentine shape with similar thickness and width.

In one embodiment, the pair of electrodes is bioresporbable and biocompatible.

In one embodiment, the pair of electrodes is formed of a bioresorbable conductive material comprising molybdenum (Mo), zinc (Zn), iron (Fe), tungsten (W), magnesium (Mg), AZ31B (3 wt % Al and 1 wt % Zn) Mg alloy, and/or other bioresorbable conductive materials.

In one embodiment, the pair of electrodes is formed without an encapsulation layer.

In one embodiment, the wireless platform comprises a power harvesting unit that operably powers the system; a near field communication (NFC) system on chip (SoC) that operably supports wireless communication; and a microcontroller unit (MCU) that operably supplies a voltage to the electrodes for stimulation and measures current between the electrodes.

In one embodiment, the wireless platform further comprises a component that serves as an audio or visual indicator of system operation.

In one embodiment, the visual indicator of system operation is a light-emitting diode (LED).

In one embodiment, the wireless power harvesting unit comprises an antenna for delivering the power to the system.

In one embodiment, the power harvesting unit operates by inductive coupling at a resonance frequency in a range of about 10-15 Mhz, preferably about 13.56 MHz.

In one embodiment, the electrotherapy system further comprises an encapsulation structure encapsulating the wireless platform.

In one embodiment, the encapsulation structure is formed of silicone elastomer, polymer, and/or dielectric materials.

In one embodiment, the electrotherapy system is adapted for electrotherapeutically treating the wound and monitoring the processes of wound healing, when the region of interest is a wound site of the subject.

In one embodiment, the pair of electrodes is designed to support levels of electrical conductivity and interface impedances that are necessary for electrotherapy and wound monitoring over several weeks of use in a thin, flexible, and stretchable construct that naturally bioresorbs into the healed tissue to eliminate the need for surgical retrieval.

In one embodiment, the inner electrode is placed on adipose tissue of the wound and the outer electrode is placed on the epidermis to mimic or reproduce in vivo conditions.

In one embodiment, the inner electrode is fixed on the wound site by a sutured splint ring structure.

In one embodiment, the inner electrode is eliminable completely from the wound site of the subject through natural chemical/biochemical processes of hydrolysis and/or metabolic actions, over a subsequent timeframe following completion of electrotherapy.

In one embodiment, the voltage is applied to the electrodes for electrostimulation for predetermined periods of time every day until full wound closure.

In one embodiment, the predetermined periods of time is customizable.

In one embodiment, the current measured during the stimulation is accociated with a dying process of the wound and provides an estimate of the healing progress, as a signature of which is drying of the wound.

In one embodiment, a gradual decrease of the current relates directly to progressive healing of the wound, and the currently gradually decreases to 0 when the wound is fully dry. In one embodiment, the electrostimulation results in an inward direct current from the healthy site to the wounded area that mimics naturally driven endogenous wound currents. In one embodiment, the electrostimulation results in acceleration of closure rate and granulation tissue formation of the wound; and/or promotion of re-epithelialization and angiogenesis in the wound.

In one embodiment, the electrostimulation reduces the times for closure of excisional splinted wounds by about 30% or more, compared to those of control and untreated groups. In one embodiment, the electrostimulation results in a transition from an early inflammation stage to the next stage by subduing the pro-inflammatory and stimulating the anti-inflammatory response.

In one embodiment, the electrotherapy system further comprises a releasable flexible connector electrically connected between the stimulator and the wireless platform.

In one embodiment, the flexible connector is configured to allow the wireless platform to be positioned onto healthy skin nearby the wound site.

In one embodiment, the stimulator and the wireless platform are directly connected to each other.

In one embodiment, the electrotherapy system further comprises a customized app with a graphical user interface operating on an external device, configured to support real-time control over stimulation parameters, serve as an interface to record the current for monitoring the progress of wound healing.

In one embodiment, the external device is a mobile device, a computer, or a cloud service.

In one embodiment, the electrotherapy system further comprises means for drug delivery, biochemical/biophysical sensing, and/or closed-loop control of operational parameters.

In another aspect of the invention, the electrotherapy system comprises a stimulator coupled with a region of interest of a subject for providing electrostimulation thereto.

In one embodiment, the stimulator comprises a pair of electrodes having a first electrode attached to the region of interest and a second electrode surrounding the first electrode.

In one embodiment, the pair of electrodes is bioresporbable and biocompatible.

In one embodiment, the electrotherapy system further comprises a microcontroller unit configured to supply a voltage to the electrodes and measure current between the electrodes.

In one embodiment, the electrotherapy system further comprises a wireless power harvesting unit configured to deliver power via resonant inductive coupling to the microcontroller unit; and a near field communication (NFC) system on chip (SoC) that operably supports wireless communication.

In one embodiment, the voltage is applied to the electrodes such that electrostimulation results in an inward direct current from the healthy site to the wounded area that mimics naturally driven endogenous wound currents.

In one embodiment, the electrotherapy system further comprises a customized app with a graphical user interface operating on an external device, configured to support real-time control over stimulation parameters, serve as an interface to record the current for monitoring the progress of wound healing.

In one embodiment, the electrotherapy system is a bioresorbable, wireless, and battery-free electrotherapy system.

In yet another aspect, the invention relates to a method for electrotherapeutic stimulation to a region of interest of a subject. The method includes providing a pair of electrodes having an inner electrode attached onto the region of interest and an outer electrode surrounding the inner electrode; and applying a voltage to the pair of electrodes for electrostimulation to the region of interest for predetermined periods of time every day until full wound closure. The region of interest is a wound site of the subject.

In one embodiment, said applying the voltage to the pair of electrodes comprises wirelessly transmitting power to a microcontroller unit by a wireless power harvesting unit via resonant inductive coupling; and applying the voltage from the microcontroller unit to the pair of electrodes that is electrically connected to the microcontroller unit.

In one embodiment, the power harvesting unit operates by resonant inductive coupling at a resonance frequency in a range of about 10-15 Mhz, preferably about 13.56 MHz.

In one embodiment, the electrostimulation results in an inward direct current from the healthy site to the wounded area that mimics naturally driven endogenous wound currents.

In one embodiment, the method of claim 46, further comprises measuring current between the inner electrode and the outer electrode; and estimating the healing progress of the wound from the current measured during the stimulation. The current is associated with a dying process of the wound.

In one embodiment, a gradual decrease of the current relates directly to progressive healing of the wound, and the currently gradually decreases to 0 when the wound is fully dry.

In one embodiment, the electrostimulation results in an electric field strength of about 1 mV/mm or more near the inner electrode and in regions of adipose tissue between the outer and inner electrodes, which is sufficient to cause migration of human keratinocyte cells to accelerate wound healing processes.

In one embodiment, the electrostimulation results in acceleration of closure rate and granulation tissue formation of the wound; and/or promotion of re-epithelialization and angiogenesis in the wound.

In one embodiment, the electrostimulation reduces the times for closure of excisional splinted wounds by about 30% or more, compared to those of control and untreated groups.

In one embodiment, the electrostimulation results in a transition from an early inflammation stage to the next stage by subduing the pro-inflammatory and stimulating the anti-inflammatory response.

In one embodiment, the inner electrode is eliminable completely from the wound site of the subject through natural chemical/biochemical processes of hydrolysis and/or metabolic actions, over a subsequent timeframe following completion of electrotherapy.

In one embodiment, the method further comprises delivering one or more drugs to the wound site; detecting biochemical/biophysical parameters associated with the wound site; and/or performing closed-loop control of operational parameters.

These and other aspects of the invention will become apparent from the following description of the preferred embodiment taken in conjunction with the following drawings, although variations and modifications therein may be affected without departing from the spirit and scope of the novel concepts of the disclosure.

### BRIEF DESCRIPTION OF THE DRAWINGS

The accompanying drawings illustrate one or more embodiments of the invention and together with the written description, serve to explain the principles of the invention. Wherever possible, the same reference numbers are used throughout the drawings to refer to the same or like elements of an embodiment.

FIG. 1 shows materials and designs of a bioresorbable, wireless, and battery-free electrotherapy system according to embodiments of the invention. Panel A: Schematic illustrations of a transient, wireless, battery-free system for electrotherapy mounted on a wound on the foot (left) and in an enlarged view (right) that highlights the different components. Panel B: Operational diagram of the entire system. Panel C: FEA results of the electric field between the positive (+) and negative (−) electrodes (scale bar=3 mm). Panel D: Schematic illustrations of the mode of use; device on a wound (i) before and (ii) after healing; (iii) removed by cutting the traces to the anode; (iv) partially bioresorbed after a period of therapy and (v) fully bioresorbed; the semi-transparent orange color represents healed skin. Panel E: Chemical reactions that govern the reaction of Mo and MOO3 with water. Panel F: Photographs of dissolution of an inner electrode structure with interconnects, captured at different times of immersion in DPBS (pH 7.4) at 75° C. (scale bar=5 mm). Panel G: Dissolution rates for a Mo electrode calculated by ICP-MS analysis of dissolved Mo ions (n=9, individual samples) and for a Mo foils determined by weight loss (n=4, individual samples).

FIG. 2 shows electrical and physical properties of bioresorbable electrodes at physiological conditions according to embodiments of the invention. Panel A: Cyclic voltammogram of a pair of Mo electrodes as an electrical stimulator during immersion in DPBS (pH 7.4) at room temperature. The inset image represents the working electrode (red) and counter electrode (black). Panel B: Changes in resistances of Mo electrodes (positive and negative) with an applied voltage of 1.1 V for 30 min/day during immersion in DPBS (pH 7.4) at 37° C. Panel C: Potentiodynamic polarization curves for positive and negative Mo electrodes after 20 min of open circuit potential (OCP) measurement during immersion in DPBS at pH 7.4. Panel D: XPS data from the surfaces of the electrodes. Panel E: Optical surface profiles of different positions on a positive Mo electrode showing (i) dense Mo oxide flakes and (ii) partially exfoliated flakes. Blue indicates Mo; green and red indicate Mo oxide. Similar profiles for the (iii) negative electrode and (iv) pristine Mo (scale bar=100 μm).

FIG. 3 shows characterization of thermal, mechanical, and electrochemical properties at in vivo conditions according to embodiments of the invention. Panel A: Schematic illustration of a wireless electrotherapy system for in vivo experiments. Panel B: Simulated electromagnetic field distribution at the central plane of a cage (dimensions, 30 cm (length)×15 cm (width)×15 cm (height)) surrounded by a double-loop antenna at heights of 3 cm and 9 cm. Panel C: Position dependent stimulation voltage across the cage at a height of 4.5 cm. Panel D: Infrared images of temperature changes after operating the device on a mouse for (i) 0, (ii) 30 min. (scale bar=10 mm). Panel E: Simulated strain-force curves associated with loading and unloading Mo electrodes over different ranges of strain, from 10, 20, and 30%. Panel F: Computed (i) shear and (ii) normal stress distributions on the skin. The color indicates stresses on the skin induced by stretching the electrodes by about 9% (scale bar=3 mm). Panel G: Cyclic voltammogram (100 mV/s, scan rate) and chronoamperometry curves of a Mo device for 15 min on the wound.

FIG. 4 shows electrostimulation accelerates wound closure in diabetic animals according to embodiments of the invention. Panel A: Digital images of wound closure. The yellow dotted line highlights the perimeter edges of the wound in each image. The inner negative electrode is embedded in the wound during the healing process and is overlaid by a white dot line. The inner diameter of splint=10 mm. Panel B: Average detected current measurements correlate with wound closure in diabetic animals. Panel C: Quantification of wound closure for day 18 days post wounding (9≤n≤11; p **<0.01; p ***<0.001). Panel D: Summary of the complete wound-closure times (5≤n≤11; p ***<0.001).

FIG. 5 shows electrostimulation facilitates pro-regenerative processes in the wound according to embodiments of the invention. Panel A: Digital images of H&E stained-tissue at day 18 post wounding. The yellow dotted line highlights the perimeter of the wound. Panel B: Masson's trichrome-stained tissue at day 30 post wounding. Quantification of (panel C) the granulation tissue thickness at day 18 post wounding (panel D) the epidermis layer at day 30 post wounding. Panel E: Epithelial differentiation visualized by cytokeratin-10 immunofluorescence staining. Panel F: angiogenesis visualized by CD31 and a-SMA immunofluorescence staining. Arrows point to the lumen of the newly formed vessels. Panel G: macrophage marker visualized by F4/80 immunofluorescence staining. Panel H: a pro-inflammatory marker visualized by IL-6 immunofluorescence staining. Panel I: an anti-inflammatory marker visualized by IL-10 immunofluorescence staining. Quantification of (panel J) the keratin-10, (panel K) the neovascularization, (panel L) the F4/80, (panel M) the IL-6, and (panel N) the IL-10. Panels A-B, E-I: (i), (ii), and (iii) indicate control, untreated, and treated groups, respectively. Panels K-N: C, U, and T represent control, untreated, treated groups, respectively. All data are represented as mean±SD. p*<0.05, p **<0.01, and p ***<0.001. Panels B, E-I, scale bar=100 μm.

FIG. 6 shows in vivo biodegradation studies according to embodiments of the invention. Panel A: Micro-CT images indicate gradual degradation of the device. scale bar=1 cm, inset yellow scale bar=2 mm. Panel B: Histological analysis of key organ tissues week 22 post-implantation. scale bar=100 μm. Panel C: In vivo biodistribution of key elements by ICP-MS (n=4). All data are represented as mean±SD.

FIG. 7 shows circuit diagrams for the wireless platform according to embodiments of the invention.

FIG. 8 shows a picture of a wireless, battery-free electrotherapy system with bioresorbable stimulation electrodes (BES) according to embodiments of the invention.

FIG. 9 shows a design of Mo electrodes with filamentary serpentine traces according to embodiments of the invention.

FIG. 10 shows 3D schematic diagrams of the electrode geometries and tissue layer stacking used in the simulations according to embodiments of the invention.

FIG. 11 shows eElectrical finite element analysis of the stimulator according to embodiments of the invention. Simulated (panel a) voltage, (panel b) current density, and (panel c) temperature change in the tissue layers during operation of the stimulator.

FIG. 12 shows healing mechanism of a chronic wound by introducing electrostimulation according to embodiments of the invention. Panel a: Healing of a chronic wound can be frustrated due to prolonged inflammation and impaired homeostasis, which causes abnormal electrical signals. Panel b: The device creates an electric field to activate migration of keratinocytes. Panel c: The inner electrode gradually disappears as it is surrounded by newly generated tissue. Panel d: Complete bioresorption after re-epithelialization and maturation.

FIG. 13 shows the biocompatibility of bioresorbable electrodes according to embodiments of the invention. Tests of the biocompatibility of Mo electrodes in live/dead staining assays of healthy mouse fibroblasts (L929) after 96 hours of culture; (panel a) on tissue culture polystyrene (TCPS) as the positive control; (b) DPBS without L929 as the negative control; (panel c) with the Mo electrode without stimulation as the untreated group; (panel d) with the Mo electrode with stimulation as the treated group. scale bar=500 μm. Panel e: Normalized in vitro assay data. n=3 independent samples. All data are represented as mean±SD.

FIG. 14 shows a dissolution study of Mo electrodes at physiological condition according to embodiments of the invention. Time-dependent concentration of Mo ions in aqueous solution from dissolution of Mo, measured by ICP-MS (n=9, individual samples) and weight loss from 1 cm2 Mo samples (n=4, individual samples). All data are represented as mean±SD.

FIG. 15 shows cyclic voltammogram of a pair of Mo electrodes as an electrical stimulator during immersion in pH 9.0 and 8.0 buffer at room temperature according to embodiments of the invention.

FIG. 16 shows experimental conditions of the bioresorbable test according to embodiments of the invention. Panel a: Design of Mo electrodes with serpentine stretchable traces for tests of electrical degradation. Panel b: Schematic illustration of the test environment without a polyimide (PI) top cover film.

FIG. 17 shows electrical properties of the bioresorbable electrode at physiological conditions according to embodiments of the invention. Changes in resistance of a Mo cathode (−) (blue) with an applied voltage of 1.1 V for 30 min/day and a Mo electrode without applied bias (black) during immersion in DPBS (pH 7.4) at 37° C.

FIG. 18 shows two different cases of corrosion behavior of the bioresorbable electrode at physiological conditions according to embodiments of the invention. Pictures of (panel a) pitting corrosion and (panel b) fracture of a dissolved Mo electrode during immersion in DPBS (pH 7.4) at 37° C. for 4 weeks.

FIG. 19 shows time-dependent oxidation of Mo foil at physiological conditions according to embodiments of the invention. (panel a) Surface and (panel b) cross-sectional colorized scanning electron microscope (SEM) images of a Mo foil immersed in DPBS (pH 7.4) at 37° C. after (i) day 0, (ii) day 3, (iii) day 15, and (iv) day 30. The blue color highlights the formation of Mo oxide on the surface. white scale bar=20 μm, yellow scale bar=2 μm, black scale bar=10 μm.

FIG. 20 shows changes in impedance at 1 KHz of Mo electrodes for stimulation at 1.1 V for 30 min/day during immersion in DPBS (pH 7.4) at 37° C., according to embodiments of the invention.

FIG. 21 shows a magnetic field distribution in the cage according to embodiments of the invention, where the arrows indicate the direction and the colors indicate magnitude.

FIG. 22 shows a thermal stability test of the BES on fat and muscle tissues according to embodiments of the invention. Panel a: Photograph of a device on fat tissue. Infrared image of the device and fat tissue during operation at (panel b) 0 s, and after (panel c) 300 s. Panel d: Photograph of the device on muscle tissue. Infrared image of the device and muscle tissue during operation at (panel e) 0 s, and after (panel f) 300 s.

FIG. 23 shows a photograph of the BES on an excisional wound according to embodiments of the invention.

FIG. 24 shows mechanical finite element analysis of the bioresorbable electrodes and electrical properties under different strain conditions according to embodiments of the invention. Finite element analysis (FEA) results for the stimulation electrodes under (panel a) 0%, 4%, and 7% biaxial stretching and (panel b) 0%, 5%, and 9% uniaxial stretching. The color in a-b represent the equivalent strain. Panel c: Changes in resistance of the Mo electrode under different stretching conditions. FEA results of (panel d) bending and (panel e) twisting of Mo electrodes under different conditions. The color represents the equivalent strain.

FIG. 25 shows the device performance was stable after 30 minutes of walking and 30 minutes of jogging when fixed to the base of the foot, fixed with a protective dressing, and covered with a sock and a running shoe.

FIG. 26 shows in vivo impedance analysis of the bioresorbable electrodes according to embodiments of the invention. Panel a: Frequency-dependent impedance between the Mo electrodes for both positive and negative electrodes on an excisional wound (black line), for only the negative electrode on an excisional wound (red line), and for both electrodes on the skin (blue line), and (panel b) corresponding experimental images. Panel c: The resistance between the Mo electrodes is 2.889 MΩ when only the negative electrode is on an excisional wound. Panel d: The resistance of between the Mo electrodes is 339.8 kΩ when both Mo electrodes are on an excisional wound.

FIG. 27 shows the study of primary human keratinocytes migration according to embodiments of the invention. Primary human keratinocytes migration was accelerated by electrostimulation. Panel a: In vitro electrotherapy system. Panel b: Area where the studies were performed (panel c) pictures of scratch assay. Panel d: quantification of migration (n=4). All data are represented as mean±SD.

FIG. 28 shows in vivo study of impedance changes during the wound healing process according to embodiments of the invention. Panel a: Current sensor data on normal mice. Panel b: Current sensor data on diabetic mice. Panel c: The current sensor data on hydrogel until the complete dehydration. All data are represented as mean±SD.

FIG. 29 shows time-dependent weight changes of the diabetic mouse during electrostimulation (n=10, individual subjects) according to embodiments of the invention. All data are represented as mean±SD.

FIG. 30 shows two sets of wound closure raw images according to embodiments of the invention. The inner diameter of splint is 10 mm.

FIG. 31 shows schematic and optical image of the simplified transient wound healing system according to embodiments of the invention.

FIG. 32 shows magnified digital images of H&E stained tissue at day 18 post wounding (scale bar=300 μm) according to embodiments of the invention.

FIG. 33 shows Masson's trichrome staining with borderline according to embodiments of the invention. (scale bar=100 μm).

FIG. 34 shows (panel a) MRSA was cultured in control, untreated, and treated environments for 24 hours in an agar plate. (scale bar=2 cm) (panel b) There was no statistical significance in colony formation. All data are represented as mean±SD.

### DETAILED DESCRIPTION OF THE INVENTION

The invention will now be described more fully hereinafter with reference to the accompanying drawings, in which exemplary embodiments of the invention are shown. This invention may, however, be embodied in many different forms and should not be construed as limited to the embodiments set forth herein. Rather, these embodiments are provided so that this invention will be thorough and complete, and will fully convey the scope of the invention to those skilled in the art. Like reference numerals refer to like elements throughout.

The terms used in this specification generally have their ordinary meanings in the art, within the context of the invention, and in the specific context where each term is used. Certain terms that are used to describe the invention are discussed below, or elsewhere in the specification, to provide additional guidance to the practitioner regarding the description of the invention. For convenience, certain terms may be highlighted, for example using italics and/or quotation marks. The use of highlighting has no influence on the scope and meaning of a term; the scope and meaning of a term is the same, in the same context, whether or not it is highlighted. It will be appreciated that same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and in no way limits the scope and meaning of the invention or of any exemplified term. Likewise, the invention is not limited to various embodiments given in this specification.

One of ordinary skill in the art will appreciate that starting materials, biological materials, reagents, synthetic methods, purification methods, analytical methods, assay methods, and biological methods other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the invention has been specifically disclosed by preferred embodiments and optional features, modification and variation of the concepts herein disclosed may be resorted to by those skilled in the art, and that such modifications and variations are considered to be within the scope of this invention as defined by the appended claims.

Whenever a range is given in the specification, for example, a temperature range, a time range, or a composition or concentration range, all intermediate ranges and subranges, as well as all individual values included in the ranges given are intended to be included in the invention. It will be understood that any subranges or individual values in a range or subrange that are included in the description herein can be excluded from the claims herein.

It will be understood that, as used in the description herein and throughout the claims that follow, the meaning of “a”, “an”, and “the” includes plural reference unless the context clearly dictates otherwise. Thus, for example, reference to “a cell” includes a plurality of such cells and equivalents thereof known to those skilled in the art. As well, the terms “a” (or “an”), “one or more” and “at least one” can be used interchangeably herein. It is also to be noted that the terms “comprising”, “including”, and “having” can be used interchangeably.

It will be understood that when an element is referred to as being “on”, “attached” to, “connected” to, “coupled” with, “contacting”, etc., another element, it can be directly on, attached to, connected to, coupled with or contacting the other element or intervening elements may also be present. In contrast, when an element is referred to as being, for example, “directly on”, “directly attached” to, “directly connected” to, “directly coupled” with or “directly contacting” another element, there are no intervening elements present. It will also be appreciated by those of skill in the art that references to a structure or feature that is disposed “adjacent” another feature may have portions that overlap or underlie the adjacent feature.

It will be understood that, although the terms first, second, third etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer or section discussed below could be termed a second element, component, region, layer or section without departing from the teachings of the invention.

Furthermore, relative terms, such as “lower” or “bottom” and “upper” or “top,” may be used herein to describe one element's relationship to another element as illustrated in the figures. It will be understood that relative terms are intended to encompass different orientations of the device in addition to the orientation depicted in the figures. For example, if the device in one of the figures is turned over, elements described as being on the “lower” side of other elements would then be oriented on “upper” sides of the other elements. The exemplary term “lower”, can therefore, encompasses both an orientation of “lower” and “upper,” depending of the particular orientation of the figure. Similarly, if the device in one of the figures is turned over, elements described as “below” or “beneath” other elements would then be oriented “above” the other elements. The exemplary terms “below” or “beneath” can, therefore, encompass both an orientation of above and below.

It will be further understood that the terms “comprises” and/or “comprising”, or “includes” and/or “including”, or “has” and/or “having”, or “carry” and/or “carrying”, or “contain” and/or “containing”, or “involve” and/or “involving”, “characterized by”, and the like are to be open-ended, i.e., to mean including but not limited to. When used in this disclosure, they specify the presence of stated features, regions, integers, steps, operations, elements, and/or components, but do not preclude the presence or addition of one or more other features, regions, integers, steps, operations, elements, components, and/or groups thereof.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and the invention, and will not be interpreted in an idealized or overly formal sense unless expressly so defined herein.

As used in the disclosure, “around”, “about”, “approximately” or “substantially” shall generally mean within 20 percent, preferably within 10 percent, and more preferably within 5 percent of a given value or range. Numerical quantities given herein are approximate, meaning that the term “around”, “about”, “approximately” or “substantially” can be inferred if not expressly stated.

As used in the disclosure, the phrase “at least one of A, B, and C” should be construed to mean a logical (A or B or C), using a non-exclusive logical OR. As used herein, the term “and/or” includes any and all combinations of one or more of the associated listed items.

The term “flexibility” or “bendability”, as used in the disclosure, refers to the ability of a material, structure, device or device component to be deformed into a curved or bent shape without undergoing a transformation that introduces significant strain, such as strain characterizing the failure point of a material, structure, device or device component. In an exemplary embodiment, a flexible material, structure, device or device component may be deformed into a curved shape without introducing strain larger than or equal to 5%, for some applications larger than or equal to 1%, and for yet other applications larger than or equal to 0.5% in strain-sensitive regions. A used herein, some, but not necessarily all, flexible structures are also stretchable. A variety of properties provide flexible structures (e.g., device components) of the invention, including materials properties such as a low modulus, bending stiffness and flexural rigidity; physical dimensions such as small average thickness (e.g., less than 100 microns, optionally less than 10 microns and optionally less than 1 micron) and device geometries such as thin film and open or mesh geometries.

The term “bending stiffness” refers to a mechanical property of a material, device or layer describing the resistance of the material, device or layer to an applied bending moment. Generally, bending stiffness is defined as the product of the modulus and area moment of inertia of the material, device or layer. A material having an inhomogeneous bending stiffness may optionally be described in terms of a “bulk” or “average” bending stiffness for the entire layer of material.

The term “elastomer”, as used in the disclosure, refers to a polymeric material which can be stretched or deformed and return to its original shape without substantial permanent deformation. Elastomers commonly undergo substantially elastic deformations. Useful elastomers include those comprising polymers, copolymers, composite materials or mixtures of polymers and copolymers. Elastomeric layer refers to a layer comprising at least one elastomer. Elastomeric layers may also include dopants and other non-elastomeric materials. Useful elastomers useful include, but are not limited to, thermoplastic elastomers, styrenic materials, olefenic materials, polyolefin, polyurethane thermoplastic elastomers, polyamides, synthetic rubbers, PDMS, polybutadiene, polyisobutylene, poly(styrene-butadiene-styrene), polyurethanes, polychloroprene and silicones. Exemplary elastomers include, but are not limited to, silicon containing polymers such as polysiloxanes including poly(dimethyl siloxane) (i.e., PDMS and h-PDMS), poly(methyl siloxane), partially alkylated poly(methyl siloxane), poly(alkyl methyl siloxane) and poly(phenyl methyl siloxane), silicon modified elastomers, thermoplastic elastomers, styrenic materials, olefenic materials, polyolefin, polyurethane thermoplastic elastomers, polyamides, synthetic rubbers, polyisobutylene, poly(styrene-butadiene-styrene), polyurethanes, polychloroprene and silicones. In one embodiment, a flexible polymer is a flexible elastomer.

The term “encapsulate” or “encapsulation”, as used in the disclosure, refers to the orientation of one structure such that it is at least partially, and in some cases completely, surrounded by one or more other structures. “Partially encapsulated” refers to the orientation of one structure such that it is partially surrounded by one or more other structures. “Completely encapsulated” refers to the orientation of one structure such that it is completely surrounded by one or more other structures. The invention includes devices having partially or completely encapsulated electronic devices, device components and/or inorganic semiconductor components.

Embodiments of the invention are illustrated in detail hereinafter with reference to accompanying drawings. The description below is merely illustrative in nature and is in no way intended to limit the invention, its application, or uses. The broad teachings of the invention can be implemented in a variety of forms. Therefore, while this invention includes particular examples, the true scope of the invention should not be so limited since other modifications will become apparent upon a study of the drawings, the specification, and the following claims. For purposes of clarity, the same reference numbers will be used in the drawings to identify similar elements. It should be understood that one or more steps within a method may be executed in different order (or concurrently) without altering the principles of the invention.

Chronic wounds, particularly those associated with diabetes mellitus, represent a growing threat to public health, with additional significant economic impacts. Inflammation associated with such wounds leads to abnormalities in endogenous electrical signals that impede the migration of keratinocytes needed to support the healing process. This observation motivates the treatment of chronic wounds with electrical stimulation therapy, but practical engineering challenges, difficulties in removing stimulation hardware from the wound site, and absence of means to monitor the healing process create barriers to widespread clinical use.

In view of the foregoing, this invention discloses a miniaturized, wireless, battery-free bioresorbable electrotherapy system that overcomes these challenges. In some embodiments, the electrotherapy system has been developed in the form of a first-of-its-kind small, flexible, stretchable bandage that accelerates healing by delivering electrotherapy directly to the wound site. In an animal study, the novel bandage healed diabetic ulcers 30 percent faster than in mice without the bandage. The bandage also actively monitors the healing process and then harmlessly dissolves-electrodes and all-into the body after it is no longer needed. The new device could provide a powerful tool for patients with diabetes, whose ulcers can lead to various complications, including amputated limbs or even death.

When a person develops a wound, the goal is always to close that wound as quickly as possible. Otherwise, an open wound is susceptible to infection. And, for people with diabetes, infections are even harder to treat and more dangerous. For these patients, there is a major unmet need for cost-effective solutions that really work for them. The novel bandage according to the invention is cost-effective, easy to apply, adaptable, comfortable, and efficient at closing wounds to prevent infections and further complications.

In addition, the active components of the novel bandage that interface with the wound bed are entirely resorbable. As such, the materials disappear naturally after the healing process is complete, thereby avoiding any damage to the tissue that could otherwise be caused by physical extraction.

By restoring or promoting a more normal electrical environment across the wound by electrostimulation with the novel bandage, cells rapidly migrated into the wound and regenerated skin tissue in the area are observed. The new skin tissue included new blood vessels, and inflammation is subdued.

In some embodiments, one side of the smart regenerative electrotherapy system contains two electrodes: a tiny flower-shaped electrode that sits right on top of the wound bed and a ring-shaped electrode that sits on healthy tissue to surround the entire wound. The other side of the electrotherapy system contains an energy-harvesting coil to power the system and a near-field communication (NFC) system to wirelessly transport data in real time. Furthermore, the electrotherapy system also includes sensors that can assess how well the wound is healing. By measuring the resistance of the electrical current across the wound, physicians can monitor progress. A gradual decrease of current measurement relates directly to the healing process. So, if the current remains high, then physicians know something is wrong.

By building in these capabilities, the device can be operated remotely without wires. From afar, a physician can decide when to apply the electrical stimulation and can monitor the wound's healing progress. As a wound tries to heal, it produces a moist environment. Then, as it heals, it should dry up. Moisture alters the current, so we are able to detect that by tracking electrical resistance in the wound. Then, we can collect that information and transmit it wirelessly. With wound care management, we ideally want the wound to close within a month. If it takes longer, that delay can raise concerns.

In a study of animal models, the electrical stimulation is applied to the wound site for just 30 minutes a day. Even this short amount of time accelerate the wound closure by 30 percent.

When the wound is healed, the flower-shaped electrode simply dissolves into the body, bypassing the need to retrieve it. In some embodiment, the electrodes are made from a metal called molybdenum, which is widely used in electronic and semiconductor applications. When molybdenum is thin enough, it can biodegrade. Furthermore, it does not interfere with the healing process.

To the best knowledge of the inventors, it is the first to show that molybdenum can be used as a biodegradable electrode for wound healing. After about six months, most of it is gone. And there is very little accumulation in the organs.

Specifically, in one aspect of the invention, the electrotherapy system comprises a pair of electrodes coupled with a region of interest of a subject for providing electrostimulation thereto; and a wireless platform coupled with the pair of electrodes for operable providing power to the stimulator.

In some embodiments, the pair of electrodes has a first electrode attached onto the region of interest and a second electrode surrounding the first electrode.

In some embodiments, the pair of electrodes is spatially apart from each other to define an electrode spacing that is larger than 1 mm.

In some embodiments, the first electrode is an inner electrode placed at the center of the region of interest and the second electrode is an outer electrode placed slightly outside of the region of interest around its perimeter.

In some embodiments, the first electrode and the second electrode are concentrically arranged such that the pair of electrodes is a concentric pair of electrodes.

In some embodiments, each of the pair of electrodes is formed with a filamentary serpentine layout so that each electrode is mechanical flexible and stretchable.

In some embodiments, each electrode has a thickness in a range of 10-30 μm and a width in range of 50-200 μm.

In some embodiments, the first electrode includes a serpentine trace with a thickness of 15 μm and width of 120 μm in a flower-like design; and the second electrode adopts a similar serpentine shape with similar thickness and width.

In some embodiments, the pair of electrodes is bioresporbable and biocompatible. In some embodiments, the pair of electrodes is formed of a bioresorbable conductive material comprising molybdenum (Mo), zinc (Zn), iron (Fe), tungsten (W), magnesium (Mg), AZ31B (3 wt % Al and 1 wt % Zn) Mg alloy, and/or other bioresorbable conductive materials. In some embodiments, the pair of electrodes is formed without an encapsulation layer. In some embodiments, the wireless platform comprises a power harvesting unit that operably powers the system; a near field communication (NFC) system on chip (SoC) that operably supports wireless communication; and a microcontroller unit (MCU) that operably supplies a voltage to the electrodes for stimulation and measures current between the electrodes.

In some embodiments, the wireless platform further comprises a component that serves as an audio or visual indicator of system operation.

In some embodiments, the visual indicator of system operation is a light-emitting diode (LED).

In some embodiments, the wireless power harvesting unit comprises an antenna for delivering the power to the system.

In some embodiments, the power harvesting unit operates by inductive coupling at a resonance frequency in a range of about 10-15 Mhz, preferably about 13.56 MHz.

In some embodiments, the electrotherapy system further comprises an encapsulation structure encapsulating the wireless platform.

In some embodiments, the encapsulation structure is formed of a silicone elastomer, polymer, and/or dielectric materials.

In some embodiments, the electrotherapy system is adapted for electrotherapeutically treating the wound and monitoring the processes of wound healing, when the region of interest is a wound site of the subject.

In some embodiments, the pair of electrodes is designed to support levels of electrical conductivity and interface impedances that are necessary for electrotherapy and wound monitoring over several weeks of use in a thin, flexible, and stretchable construct that naturally bioresorbs into the healed tissue to eliminate the need for surgical retrieval.

In some embodiments, the inner electrode is placed on adipose tissue of the wound and the outer electrode is placed on the epidermis to mimic or reproduce in vivo conditions.

In some embodiments, the inner electrode is fixed on the wound site by a sutured splint ring structure.

In some embodiments, the inner electrode is eliminable completely from the wound site of the subject through natural chemical/biochemical processes of hydrolysis and/or metabolic actions, over a subsequent timeframe following completion of electrotherapy.

In some embodiments, the voltage is applied to the electrodes for electrostimulation for predetermined periods of time every day until full wound closure.

In some embodiments, the predetermined periods of time is customizable.

In some embodiments, the current measured during the stimulation is associated with a dying process of the wound and provides an estimate of the healing progress, as a signature of which is drying of the wound.

In some embodiments, a gradual decrease of the current relates directly to progressive healing of the wound, and the currently gradually decreases to 0 when the wound is fully dry.

In some embodiments, the electrostimulation results in an inward direct current from the healthy site to the wounded area that mimics naturally driven endogenous wound currents. In some embodiments, the electrostimulation results in acceleration of closure rate and granulation tissue formation of the wound; and/or promotion of re-epithelialization and angiogenesis in the wound.

In some embodiments, the electrostimulation reduces the times for closure of excisional splinted wounds by about 30% or more, compared to those of control and untreated groups.

In some embodiments, the electrostimulation results in a transition from an early inflammation stage to the next stage by subduing the pro-inflammatory and stimulating the anti-inflammatory response.

In some embodiments, the electrotherapy system further comprises a releasable flexible connector electrically connected between the stimulator and the wireless platform.

In some embodiments, the flexible connector is configured to allow the wireless platform to be positioned onto healthy skin nearby the wound site.

In some embodiments, the stimulator and the wireless platform are directly connected to each other.

In some embodiments, the electrotherapy system further comprises a customized app with a graphical user interface operating on an external device, configured to support real-time control over stimulation parameters, serve as an interface to record the current for monitoring the progress of wound healing.

In some embodiments, the external device is a mobile device, a computer, or a cloud service.

In some embodiments, the electrotherapy system further comprises means for drug delivery, biochemical/biophysical sensing, and/or closed-loop control of operational parameters. In another aspect of the invention, the electrotherapy system comprises a stimulator coupled with a region of interest of a subject for providing electrostimulation thereto. In some embodiments, the stimulator comprises a pair of electrodes having a first electrode attached to the region of interest and a second electrode surrounding the first electrode.

In some embodiments, the pair of electrodes is bioresporbable and biocompatible. In some embodiments, the electrotherapy system further comprises a microcontroller unit configured to supply a voltage to the electrodes and measure current between the electrodes.

In some embodiments, the electrotherapy system further comprises a wireless power harvesting unit configured to deliver power via resonant inductive coupling to the microcontroller unit; and a near field communication (NFC) system on chip (SoC) that operably supports wireless communication.

In some embodiments, the voltage is applied to the electrodes such that electrostimulation results in an inward direct current from the healthy site to the wounded area that mimics naturally driven endogenous wound currents.

In some embodiments, the electrotherapy system further comprises a customized app with a graphical user interface operating on an external device, configured to support real-time control over stimulation parameters, serve as an interface to record the current for monitoring the progress of wound healing.

In some embodiments, the electrotherapy system is a bioresorbable, wireless, and battery-free electrotherapy system.

In yet another aspect, the invention relates to a method for electrotherapeutic stimulation to a region of interest of a subject. The method includes providing a pair of electrodes having an inner electrode attached onto the region of interest and an outer electrode surrounding the inner electrode; and applying a voltage to the pair of electrodes for electrostimulation to the region of interest for predetermined periods of time every day until full wound closure. The region of interest is a wound site of the subject.

In some embodiments, said applying the voltage to the pair of electrodes comprises wirelessly transmitting power to a microcontroller unit by a wireless power harvesting unit via resonant inductive coupling; and applying the voltage from the microcontroller unit to the pair of electrodes that is electrically connected to the microcontroller unit.

In some embodiments, the power harvesting unit operates by resonant inductive coupling at a resonance frequency in a range of about 10-15 Mhz, preferably about 13.56 MHz.

In some embodiments, the electrostimulation results in an inward direct current from the healthy site to the wounded area that mimics naturally driven endogenous wound currents.

In some embodiments, the method of claim 46, further comprises measuring current between the inner electrode and the outer electrode; and estimating the healing progress of the wound from the current measured during the stimulation. The current is associated with a dying process of the wound.

In some embodiments, a gradual decrease of the current relates directly to progressive healing of the wound, and the currently gradually decreases to 0 when the wound is fully dry.

In some embodiments, the electrostimulation results in an electric field strength of about 1 mV/mm or more near the inner electrode and in regions of adipose tissue between the outer and inner electrodes, which is sufficient to cause migration of human keratinocyte cells to accelerate wound healing processes.

In some embodiments, the electrostimulation results in acceleration of closure rate and granulation tissue formation of the wound; and/or promotion of re-epithelialization and angiogenesis in the wound.

In some embodiments, the electrostimulation reduces the times for closure of excisional splinted wounds by about 30% or more, compared to those of control and untreated groups.

In some embodiments, the electrostimulation results in a transition from an early inflammation stage to the next stage by subduing the pro-inflammatory and stimulating the anti-inflammatory response.

In some embodiments, the inner electrode is eliminable completely from the wound site of the subject through natural chemical/biochemical processes of hydrolysis and/or metabolic actions, over a subsequent timeframe following completion of electrotherapy.

In some embodiments, the method further comprises delivering one or more drugs to the wound site; detecting biochemical/biophysical parameters associated with the wound site; and/or performing closed-loop control of operational parameters.

These and other aspects of the invention are further described below. Without intent to limit the scope of the invention, exemplary instruments, apparatus, methods and their related results according to the embodiments of the invention are given below. Note that titles or subtitles may be used in the examples for convenience of a reader, which in no way should limit the scope of the invention. Moreover, certain theories are proposed and disclosed herein;

however, in no way they, whether they are right or wrong, should limit the scope of the invention so long as the invention is practiced according to the invention without regard for any particular theory or scheme of action.

### Example

### Bioresorbable, Wireless, and Battery-Free System for Electrotherapy and Impedance Sensing at Wound Sites

Chronic wounds, particularly those associated with diabetes mellitus, represent a growing threat to public health, with additional significant economic impacts. Inflammation associated with such wounds leads to abnormalities in endogenous electrical signals that impede the migration of keratinocytes needed to support the healing process. This observation motivates the treatment of chronic wounds with electrical stimulation therapy, but practical engineering challenges, difficulties in removing stimulation hardware from the wound site, and absence of means to monitor the healing process create barriers to widespread clinical use.

In this exemplary example, we demonstrate a miniaturized wireless, battery-free bioresorbable electrotherapy system overcomes these challenges. Studies based on a splinted diabetic mouse wound model confirm the efficacy for accelerated wound closure by guiding epithelial migration, modulating inflammation, and promoting vasculogenesis. Changes in the impedance provide means for tracking the healing process. The results offer potential as a simple and effective platform for wound site electrotherapy.

Specifically, the bioresorbable, wireless, and battery-free electrotherapy system (BES) provides electrostimulation and impedance measurements across a wound in a manner that avoids the aforementioned disadvantages. The stimulation process mimics naturally occurring endogenous electric fields to promote healing. The impedance data provide the basis for real-time monitoring of wound closure. The BES includes a pair of compliant electrodes that supports stable operation over several weeks and then slowly bioresorbs via hydrolysis to eliminate the need for retrieval. A miniaturized wireless, battery-free electronics module interfaces to these electrodes, with a graphical user interface that runs on a smartphone. This technology, which can be easily used in both the hospital and home settings, has the potential to improve the care of patients with chronic diabetic foot ulcers (DFUs) and with further study may provide new options to treat other skin wounds.

### Materials and Methods

### Fabrication of a Bioresorbable, Wireless, and Battery-Free Electrotherapy System (BES)

A laser cutting process applied to uniform foils of Mo (15 or 25 μm thick; Goodfellow) formed the bioresorbable metal inner and outer electrodes. Schematic diagrams and the board layouts for the wireless platform were designed using AUTODESK EAGLE (version 9.6.0). The components included 0201 inch footprint passive elements (capacitors, resistors, and Schottky diodes), seven turn coils for wireless powering (resonant frequency: 13.56 MHz), a microcontroller (ATiny84A), low dropout linear regulators (Vₒᵤₜ: 2.8V and 1.1V), a near-field communication (M24LR04E-R), amplifier (OPA330AIYFFR), and a red light-emitting diode. A silicone elastomer (Silbione-4420) formed an encapsulating structure for the wireless platform. All samples underwent sterilization by EtO gas (Anprolene® AN74i) before in vivo studies.

### Electrical and Heating Simulations

Finite element analysis was implemented on the commercial software COMSOL 5.2a by coupling the Electric Current, Heat Transfer, and Electric Circuit Modules to determine the electric field, current density, current in the electrodes, and the temperature change in the wound and tissue layers of the mouse for a voltage of 1.1V applied to the electrode.

The stationary form of the partial differential equation for the electric current is

$$∇ \cdot J = 0 \qquad ( 1 )$$

where J is the current defined as J=σE. The electric field is given by E=∇V, where σ is the electrical conductivity and V is the electrical potential in the electrode.

The heat transfer process is governed by

$$ρ {C}_{p} \cdot ∇ T = ∇ \cdot ( k ∇ T ) + {Q}_{e} \qquad ( 2 )$$

where ρ is the density, Cₚ is the specific heat capacity, k is the thermal conductivity, T is the temperature field. The Joule heating effect is introduced by the term Qₑ=J·E. The electrode terminals were connected in series to a DC voltage source and resistor R=100 kΩ through the External I vs U feature that links the physics interfaces.

The Mo electrode and the mouse tissue layers were modeled using 4-node tetrahedral elements. A convergence test of the mesh size was performed to ensure accuracy. The total number of elements in the models was approximately about 2,000,000. The thickness and material properties of the tissue layers used in the simulation are listed in Table 1. As used herein the table, the notation “[n]” represents the nth reference cited in the reference list.

TABLE 1
Electrode and Tissue Layer Properties used in the Multiphysics Simulation

| | | | Specific | | | |
|---|---|---|---|---|---|---|
| | | | Heat | | | |
| | | Density | Capacity | Thermal | Relative | Electrical |
| Electrode and | Thickness | (ρ) | (Cp) | Conductivity | Permittivity | Conductivity |
| Tissue Layers | (μm) | (kg/m³) | (J/kg K) | (k) (W/mK) | (ε) | (σ) (S/m) |
| Molybdenum | 15 | 10200 | 250 | 138 | 1 | 2E7 |
| | | [41] | [41] | [41] | [41] | [41] |
| Stratum | 5 | 1090 | 3350 | 0.209 | 5E2 | 2E−6 |
| Corneum | [42, 43] | [44] | [44] | [45] | [46, 47] | [46, 47] |
| Viable | 22 | 1090 | 3350 | 0.209 | 5E6 | 0.026 |
| Epidermis | [42, 43] | [44] | [44] | [45] | [46, 47] | [46, 47] |
| Dermis | 280 | 1090 | 3350 | 0.322 | 5E9 | 0.222 |
| | [42, 43] | [44] | [48] | [45] | [46, 47] | [46, 47] |
| Subcutaneous | 300 | 911 | 3660 | 0.21 | 5E7 | 0.08 |
| Fat (Adipose | [48, 49] | [50] | [51] | [45] | [46, 47] | [46, 47] |
| tissue + | | | | | | |
| Panniculus | | | | | | |
| Carnosus | | | | | | |
| Muscle) | | | | | | |
| Muscle | 2000 | 1060 | 3730 | 0.530 | 2E7 | 0.25 |
| | [52] | [53] | [54] | [55] | [46, 47] | [46, 47] |

### Electromagnetic Simulation

Finite element analysis was conducted for electromagnetic simulations to study the magnetic field distribution around the transmitting antenna. The simulations were performed using the commercial software Ansys HFSS, where tetrahedron elements were used in the solution with adaptive meshing convergence. An adaptive mesh convergence condition and a spherical radiation boundary (1000 mm in radius) were adopted to ensure computational accuracy. The electromagnetic parameters in the material library of Ansys HFSS were used in the simulation.

Mechanical Simulation 3D finite element analysis (FEA) was employed to design the Mo electrode and to predict the strain under stretching, bending and twisting. Eight-node 3D solid elements, and four-node shell elements were used for the tissue, and Mo electrode, respectively. Convergence of mesh sizes was tested to ensure computational accuracy using the commercial software ABAQUS. In the FEA model, the Mooney-Rivlin strain energy potential model was used for the tissue (elastic modulus Eₜᵢₛₛᵤₑ=130 kPa and Poisson's ratio Vₜᵢₛₛᵤₑ=0.49) displaying hyperelastic material behavior, where the relevant materials parameters include C10=0.0174 MPa, C01=0.0044 MPa, D1=0.923 MPa-1. Molybdenum was modeled with ideal elastoplastic behavior, where the elastic modulus, Poisson's ratio and elastic strain limit are E_{Mo}=315 GPa, ν_{Mo}=0.29 and ε_{Mo}=0.35%, respectively.

### The Diabetic Wound Healing Model

All in vivo studies were approved by the Institutional Animal Care and Use Committee (IACUC) at Northwestern University (protocol IS00000373, IS00018748). Diabetic (db/db) mice (BKS.Cg-m+/+Leprdb, #000642; homozygous for Leprdb) were purchased from Jackson Laboratory (Bar Harbor, ME, USA). A splinted excisional wound model in db/db mice (BKS.Cg-Dock7m+/+Leprdb/J Homozygous for Leprdb) was utilized, as previously described47. To prevent skin contraction, paired sterilized doughnut-shaped acrylate splints (10-mm inner diameter; 12-mm outer diameter) (3M, St. Paul, MN) were attached to the left and right dorsal sides of the mouse with Vetbond (3M) and interrupted 6-0 nylon sutures (Ethicon, Cincinnati, OH) after depilation. A 6-mm circular, the full-thickness wound was made in the center of each splinted area. For the groups with devices, each device was laminated with the inner electrode on the center of the wound and the outer electrode along the edge of the wound. A transparent sterile occlusive dressing, TegaDerm™ (3M), was then placed over the wound and the splint. The mouse was monitored every day and digital images of the wound area were taken every three days and quantified by three blinded observers using ImageJ by normalizing the wound area to the known splint area at each time point. The treated group had Mo electrodes with DC electrostimulation for 30 minutes every day until full wound closure; the untreated group had Mo electrodes without electrostimulation. The control groups did not have Mo electrodes. Also, for the treated group, the endogenous current was monitored for 5 minutes daily until there was no signal.

### Tissue Processing and Immunofluorescence Staining

For tissue processing and histology, upon 30 days, 18 days, or 4 days after creating the wound, animals were euthanized, and the regenerated wound tissue was excised with a 10-mm biopsy punch (Acuderm, Fort Lauderdale, FL), fixed using 4% paraformaldehyde and embedded by paraffin. The tissues were then sectioned and stained with hematoxylin and eosin (H&E) to measure granulation tissue thickness. The tissues were also stained with Masson's trichrome to measure the epithelial thickness. The granulation tissue and epithelial thickness were quantified by measuring the thickness at 5 evenly spaced locations from the center of the wound for each animal using ImageJ. The average of the measurements obtained from each wound was calculated and compared among treatment groups.

Moreover, the tissue sections were stained for Keratin 10, a-SMA, CD31, F4/80, IL-6, or IL-10 (Santa Cruz, Dallas, TX). The secondary antibodies were either conjugated to AlexaFluor488 or AlexaFluor555 (Invitrogen, Carlsbad, CA). Controls consisted of samples stained with the secondary antibody without incubation with a primary antibody. The development of neovascularization and changes in inflammation were quantified using ImageJ.

### Micro CT and Fundamental Organ Analysis

Mice were anesthetized with isoflurane and placed on the heated bed of the microCT system. Images were acquired with a preclinical microPET/CT imager, nanoScan scanner (Mediso-USA, Arlington, VA). Data was acquired with “medium” magnification, <60 μm focal spot, 1×1 binning, with 720 projection views over a full circle, with a 300 ms exposure time. Images were acquired using 70 kVp. The projection data were reconstructed with a voxel size of 34 μm and using filtered (Butterworth filter) backprojection software from Mediso. The reconstructed data was visualized and segmented in Amira 2020.2 (FEI, Houston, TX). For the histological analysis of essential organs (heart, lung, liver, spleen, kidney, and brain), mice were sacrificed after 2 weeks, 15 weeks, and 22 weeks of implantation. Organs were harvested and fixed using 4% paraformaldehyde and then embedded with paraffin. The organs were then sectioned and stained with hematoxylin and eosin (H&E) for qualitative analysis.

### ICP-MS Study

The process for quantifying the amount of Mo in targeted tissues used ICP-MS applied to acid samples digested by immersion trace grade nitric acid (HNO₃, >69%, Thermo Fisher Scientific, Waltham, MA, USA) and trace grade hydrogen peroxide (H₂O₂, 30.0-32.0%, GFS Chemicals, Columbus, OH, USA) and then heating to 65° C. for at least 3 hours. The addition of ultra-pure H₂O (18.2 MΩ··cm) yielded a final solution of 5.0% nitric acid. Specific volumes of nitric acid, hydrogen peroxide, and final solutions are given in Table 2 according to tissue type.

TABLE 2
Volumes of nitric acid, hydrogen peroxide, and
final solutions according to tissue type

| | Approximate | | | |
|---|---|---|---|---|
| | wt of tissue | Vol HNO₃ | Vol H₂O₂ | Final vol |
| Tissue type | used (g) | (mL) | (mL) | (mL) |
| Whole blood | 0.2 | 0.25 | 0.25 | 5 |
| Liver | 1-1.5 | 2 | 0.5 | 40 |
| All others | 0.1-0.5 | 0.5 | 0.125 | 10 |

Quantitative standards used a 1000 μg/mL Mo Standard solution (Inorganic Ventures, Christiansburg, VA, USA) to create a 100 ng/g Mo in 5.0% nitric acid (v/v) in a total sample volume of 50 mL. A solution of 5.0% nitric acid (v/v) was used as the calibration blank. ICP-MS used a computer-controlled (QTEGRA software) Thermo iCapQ ICP-MS (Thermo Fisher Scientific, Waltham, MA, USA) operating in KED mode and equipped with an ESI SC-2DX PrepFAST autosampler (Omaha, NE, USA). The internal standard was added inline using the prepFAST system and consisted of 1 ng/mL of a mixed element solution containing Bi, In, 6Li, Sc, Tb, Y (IV-ICPMS-71D from Inorganic Ventures). Online dilution was also performed by the prepFAST system and used to generate a calibration curve including 100, 50, 20, 10, 1, 0.5 ppb Mo. Each sample was acquired using 1 survey run (10 sweeps) and 3 main (peak jumping) runs (40 sweeps). The isotopes selected for analysis were 92,95,96Mo and 90Zr, 101Ru (chosen to perform interference corrections), and 89Y, 115 In (chosen as internal standards for data interpolation and machine stability). Instrument performance was optimized daily through autotuning, followed by verification via a performance report (passing manufacturer specifications).

### In Vitro Biocompatibility Test

For the in vitro viability assay, a mouse fibroblast cell line (L929) was purchased from ATTC (ATCC® CCL-1™) along with their media (ATCC® 30-2003™). Cells were maintained and cultured in T-25 flasks according to the manufacturer's protocols. After 10,000 cells were seeded onto the 24 well-plate, EtO gas sterilized electrodes were placed on top. After maintaining them for 96 hours, resazurin assay and live and dead staining were performed as the manufacturer's protocol. Resazurin salt was purchased from Sigma-Aldrich (R7017), and Live/dead viability/cytotoxicity Kit (L3224) was purchased from Invitrogen™ Both fluorescence measurement and imaging were done with Cytation5 (Biotek®).

### Keratinocyte Migration Assay

Primary human keratinocytes (KR-F) and their growth medium (KM-2) were purchased from Zen-bio for the scratch assay. Keratinocytes were maintained according to the manufacturer's protocols, and cells were used for migration assays after the first or second subculture. To electrostimulate cells, the electrodes were bonded onto a poly(dimethylsiloxane) (PDMS) substrate, and the electrodes were connected to a function generator (SDG1025, Siglent). For fabrication of these components, PDMS (Sylgard 184, Dow Corning) base was mixed with a curing agent at a weight ratio of 10:1 (base: curing agent) and poured onto a petri dish. After degassing, PDMS was semi-cured in an oven maintained at 65° C. for 15 min. The trimmed Mo electrodes were placed onto semi-cured PDMS and then fully cured. The electrode with PDMS substrate was then connected to the printed circuit board via anisotropic conductive film (Elform). After the device was EtO gas sterilized and glued to the surface of a 6-well plate, the cell-culture surface on the device was coated with fibronectin solution (0.1 mg/ml in phosphate-buffered saline (PBS)) (Corning-356008) for 2 hours incubation in a cell culture incubator at 37° C. and 5% CO₂. Next, the surface was washed with PBS and filled with culture medium prior to seeding of human keratinocytes at a concentration of 1×10⁶ cells/ml. After an hour of incubation to ensure that the cells settle onto the surface, the well was filled with a culture medium. After 24 hours, scratches were made using a 200 μl pipette tip. Using a function generator, 200 mv/mm was applied for 24 hours. Quantitative analysis was performed using ImageJ.

### Results

### Bioresorbable, Wireless, and Battery-Free Electrotherapy System (BES)

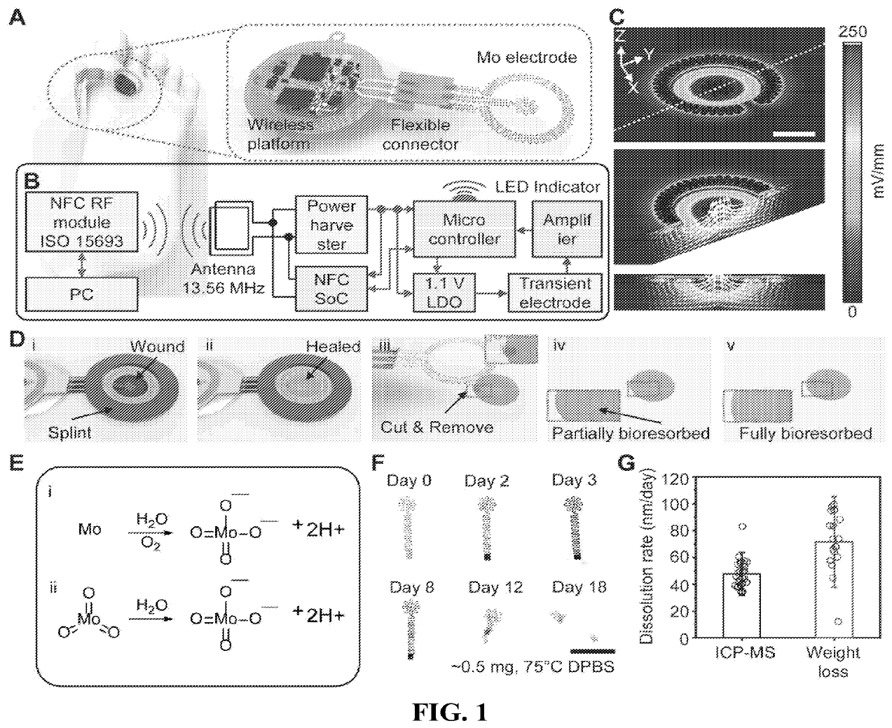

Panel A of FIG. 1 shows a schematic illustration of a BES on the surface of a wound on the foot. The right side of this frame highlights the various components, including a wireless platform, a releasable flexible connector, and a stimulator that include a concentric pair of Mo electrodes with filamentary serpentine layouts for stretchability and without an encapsulation layer.

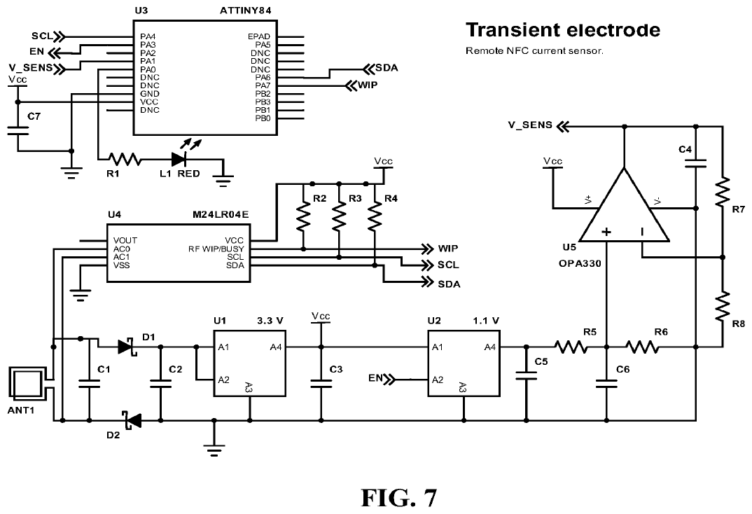

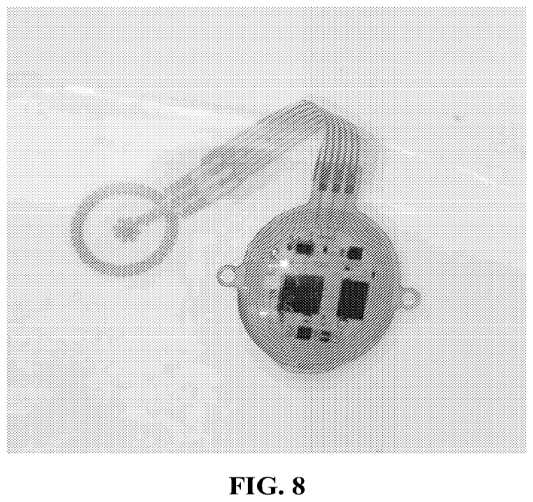

The wireless platform includes 1) a power harvesting coil that operates by magnetic inductive coupling at a resonance frequency of 13.56 MHz, to power the system, 2) an NFC system on chip (SoC) that supports wireless communication, 3) a red LED that serves as a visual indicator of system operation, and 4) a microcontroller unit (MCU) that supplies controlled voltages for stimulation and measures the applied current (panel B of FIG. 1 and FIGS. 7-8). In some embodiments, the electric current is measured with a shunt resistor, connected in series with the electrode. The voltage in the shunt resistor is amplified with an instrumentation amplifier and then digitalized with the microcontroller's ADC. The current is then calculated considering the shunt resistor value, the ADC value, and the amplification gain. The wireless device employs a customized routine to perform sampling averaging (n=10-20) on a chip. With this device option and the moderate low amplification gain set in the analog front-end module, the noise due to other factors becomes negligible.

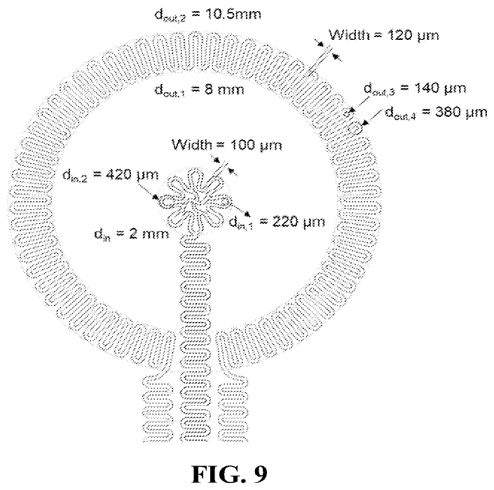

In one embodiment, the inner and outer Mo electrodes reside at the center of the wound and slightly outside of the wound around its perimeter, respectively. The thin geometries of these electrodes allow for mechanical flexibility; the serpentine layouts afford some level of stretchability. Specifically, the inner electrode includes a serpentine trace with a thickness of 15 μm and width of 120 μm in a flower-like design (dᵢₙ; diameter of 2 mm); the outer electrode adopts a similar serpentine shape with similar thickness and width (dₒᵤₜ₁; inner diameter of 8 mm, and dₒᵤₜ₂; outer diameter of 10.5 mm) (FIG. 9). The flexible connector allows the wireless platform to be positioned onto healthy skin nearby the wound site.

In one embodiment, the Mo electrodes and the wireless platform was bonded using anisotropic conductive film (Elform). The electrodes were composed of two separate parts: the outer circular and inner straight electrodes, and the resistive serpentine pattern will provide extra accommodation to the surroundings. Since the splinted excisional wound diabetic mouse model has a 6 mm, circular wound, the diameter of the outer electrode was set in between 8 mm and 10.5 mm. Thus, the external electrode can be placed at the uninjured site, and the inner electrode can be placed at the center of the wound.

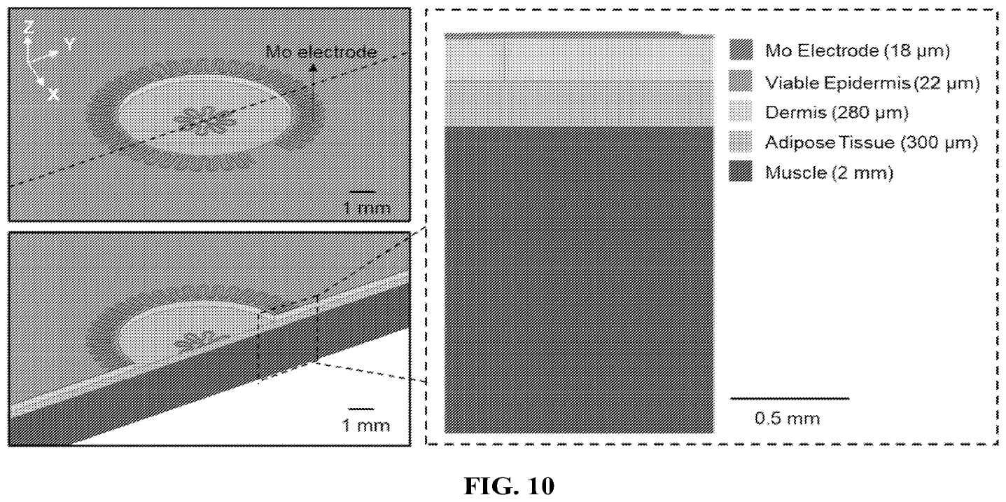

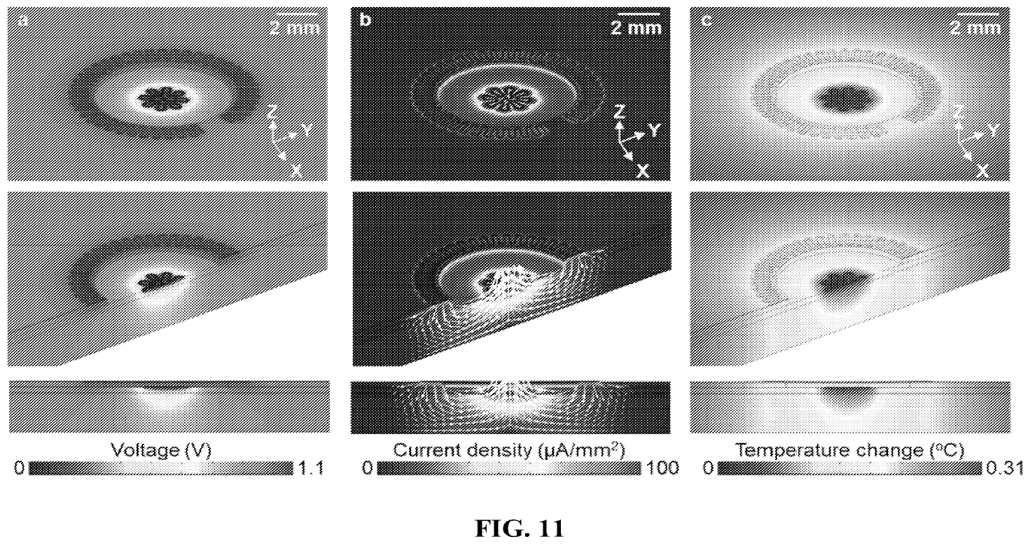

Three-dimensional finite element analysis (FEA) captures the spatial distribution of the electric field, voltage, current density, and temperature during direct current (DC) 1.1 V stimulation with the inner electrode on adipose tissue and the outer electrodes on the epidermis to mimic or reproduce in vivo conditions (panel C of FIG. 1 and FIGS. 10-11). The inward direct current from the healthy site to the wounded area mimics naturally driven endogenous wound currents. The electric field strength approaches about 250 mV/mm near the inner electrode. Other regions of adipose tissue between the outer and inner electrodes experience field strengths of about 100 mV/mm, known to be sufficient to cause migration of human keratinocyte cells to accelerate wound healing processes.

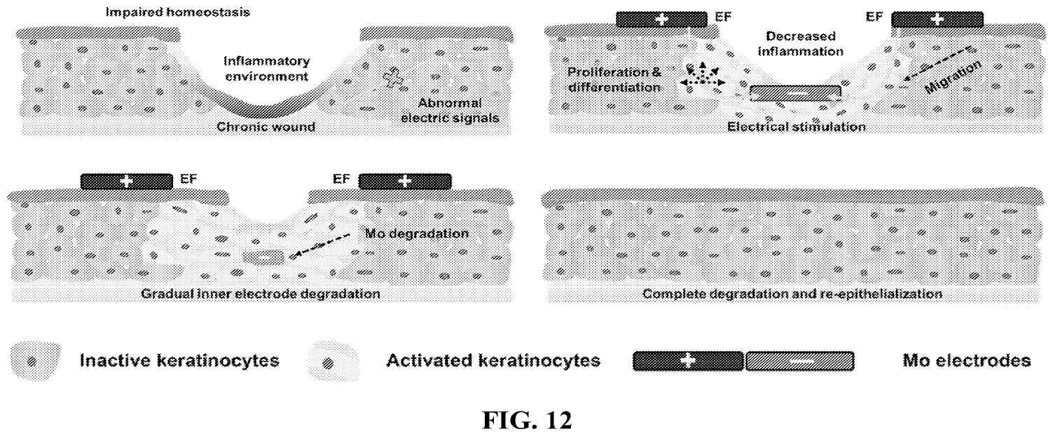

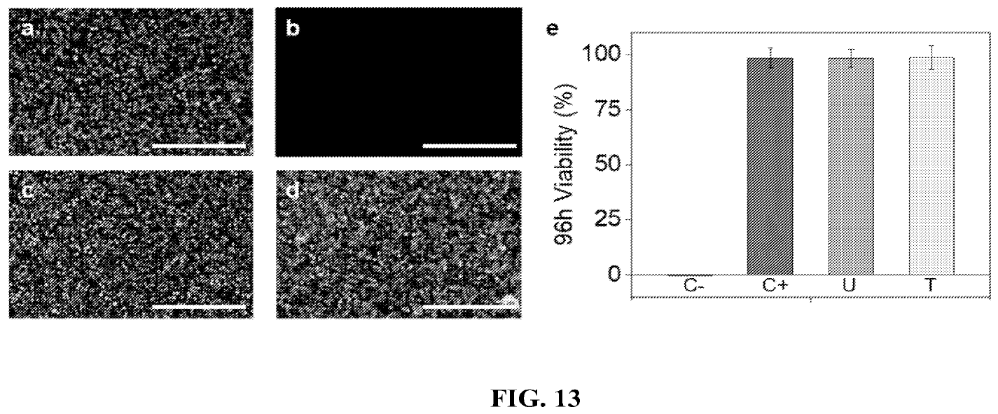

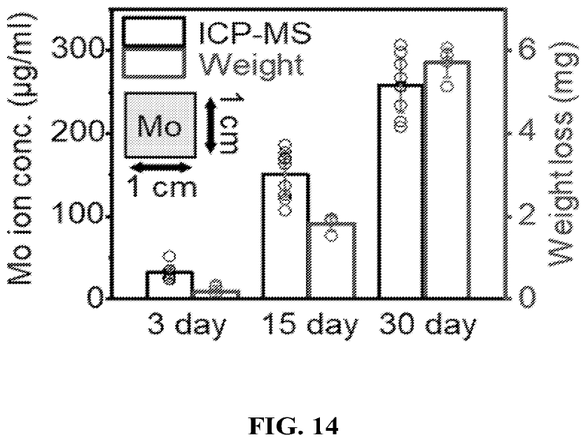

Panel D of FIG. 1 illustrates the scheme for using this system in electrotherapy. A sutured splint ring structure fixes the Mo electrodes at the site of a wound, positioned as described above (panel D-i of FIG. 1). As the wound heals, the inner Mo electrode becomes embedded in regenerated skin (panel D-ii of FIG. 1 and FIG. 12). After completing the therapy, cutting the trace that leads to this electrode allows removal of the wireless platform and the outer Mo electrode (panel D-iii of FIG. 1). The inner electrode (mass <500 μg) gradually bioresorbs into nontoxic products (Mo+2H₂O+O₂→MoO₄²⁻+4H⁺, MoO₃+H₂O→MoO₄²⁻+2H⁺) to finally disappear without a trace over a timescale of months (panel D-iv, v, and panel E of FIG. 1). In vitro tests of the Mo electrodes indicate good biocompatibility (FIG. 13). Panel F of FIG. 1 presents a sequence of images of the inner electrode structure at various points of dissolution in Dulbecco's phosphate-buffered saline (DPBS) (pH 7.4) at 75° C., corresponding to a roughly 16 times acceleration relative to body temperature. The electrode begins to lose its shape after day 12 and almost disappears after day 18. Inductive coupled plasma mass spectrometry (ICP-MS) analysis and weight loss measurements associated with dissolution of a foil of Mo (about 1 cm2) indicate a dissolution rate of 50 about 70 nm/day at 37° C. with various time points, which is consistent with previous reports and with the results of accelerated testing (panel G of FIG. 1 and FIG. 14).

### Dissolution of the Thin, Serpentine Mo Electrodes

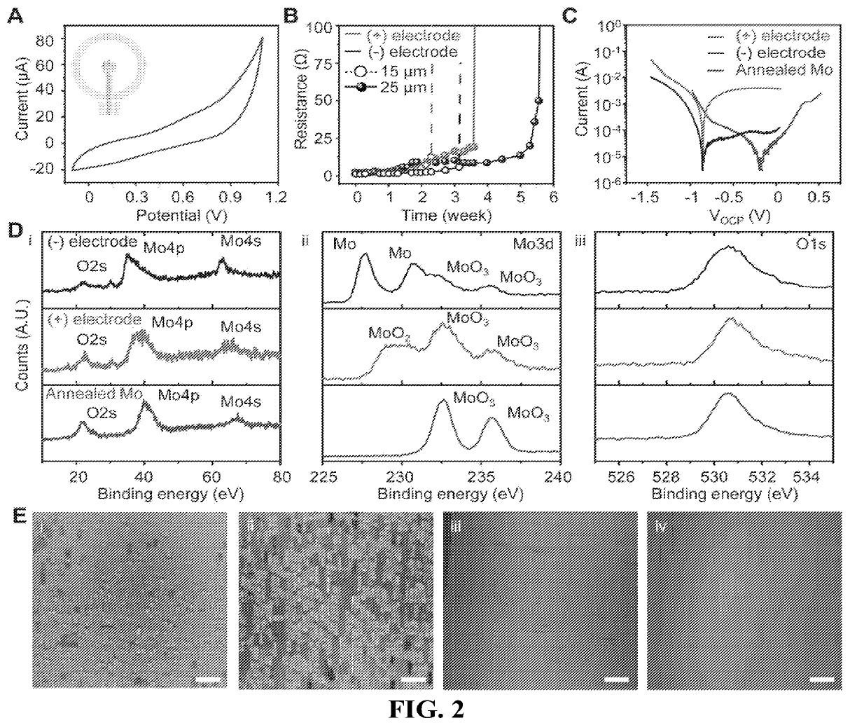

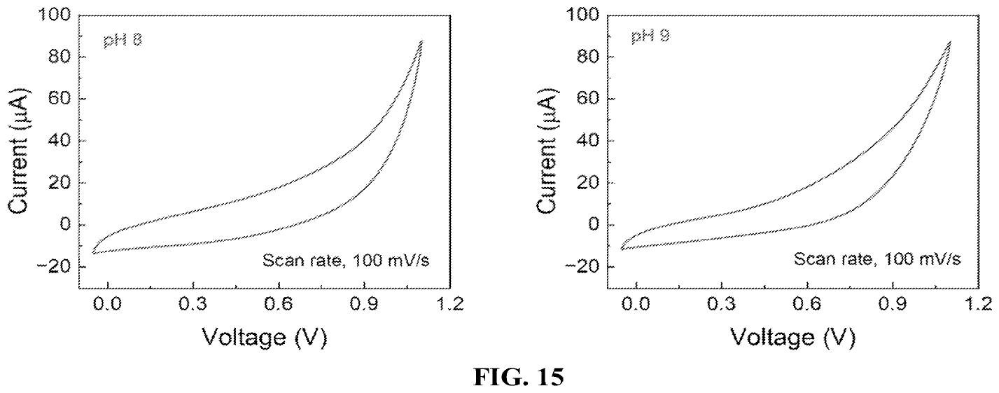

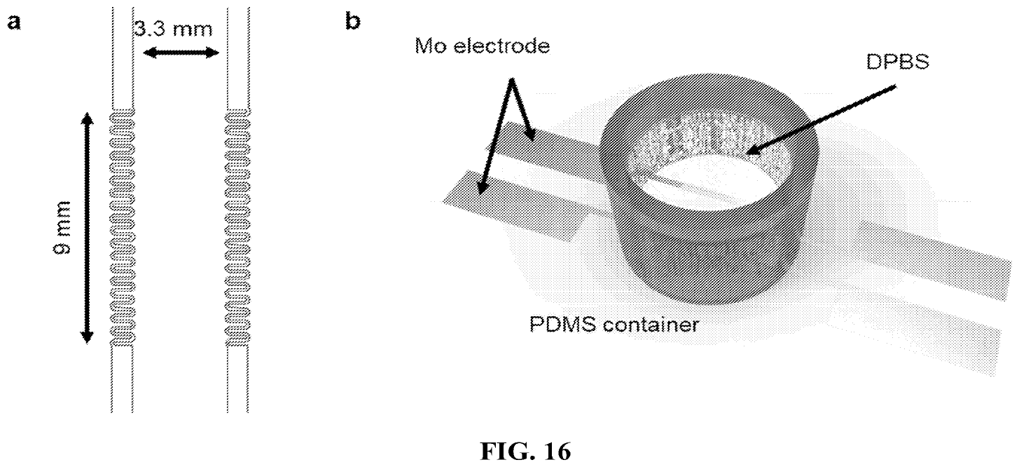

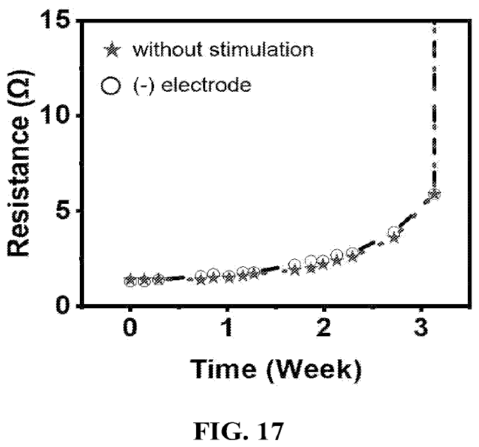

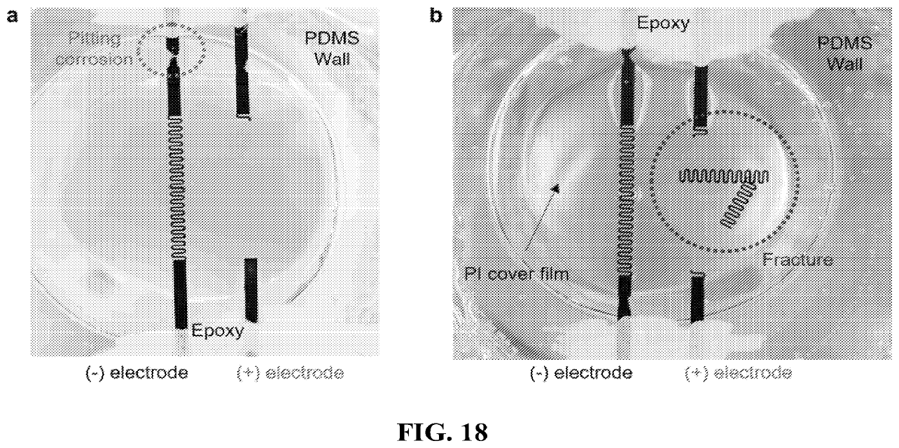

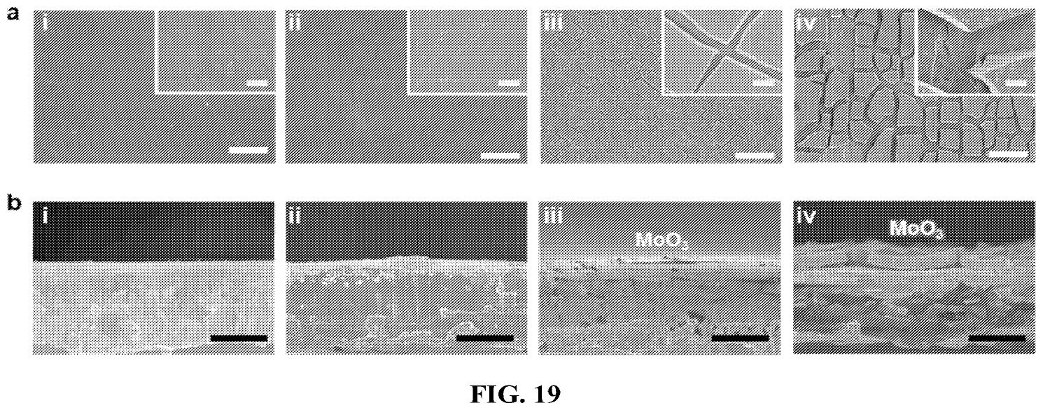

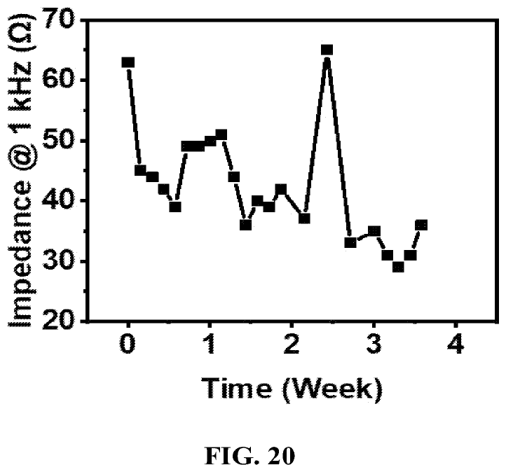

The electrical properties of the electrodes without an encapsulation layer and their stability of operation over a timeframe set by the healing process have not been previously investigated and are important to understand for this potential medical application. Cyclic voltammetry (CV) analysis of a Mo electrode immersed in DPBS (pH 7.4) at room temperature indicates no additional redox response except normal oxidation and reduction of Mo across voltages from 0 V to 1.1 V (panel A of FIG. 2). CV analysis in an alkaline environment was similar to a pH of 7.4 (FIG. 15). Tests of electrical degradation involve immersion in DPBS (pH 7.4) at 37° C. housed in a container formed in PDMS (volume of 5 ml) with a polyimide (PI) top cover film to prevent water evaporation (FIG. 16). DC stimulation causes electrolytic corrosion on the anode (+), thereby accelerating the rate of corrosion to values about 30% above those of the cathode (−), for the case of an applied DC voltage of 1.1 V for 30 minutes per day (panel B of FIG. 2). Studies of Mo electrodes with thicknesses of 15 and 25 μm confirm that functional lifetime has a linear relationship with thickness. In fact, the rate of corrosion of the cathode in this case is similar to that of an electrode without applied voltage (FIG. 17). Due to accelerated corrosion at the interface between the PDMS container and the Mo electrodes (FIG. 18), the results presented here overestimate the intrinsic rates of corrosion. The annealing process forms a thin and uniform layer of molybdenum trioxide (MoO₃) on the Mo surface, to suppress the initial dissolution rate due to relatively higher corrosion potential (−0.16 V_{corr}) than that of Mo (panels C-D of FIG. 2). After this MoO₃ passivation layer dissolves from the surface at an applied DC voltage of 1.1 V over 24 hours in DPBS at room temperature, the corrosion potentials of both anode and cathode decrease to −0.85 V_{corr}. XPS analysis indicates the formation of electrochemically grown molybdenum dioxide (MoO₂) at the anode and reduction of Mo at the cathode (panel D of FIG. 2). Optical profiler images of different anode surfaces show flakes of MoO₂ and MoO₃ that exfoliate from the Mo surface due to the high Pilling-Bedworth ratio (panel E-i, ii of FIG. 2 and FIG. 19). This process exposes fresh Mo at the anode, thereby sustaining the original surface impedance over time (FIG. 20). The cathode retains a clean surface.

### Apparatus for Wireless In Vivo Operation

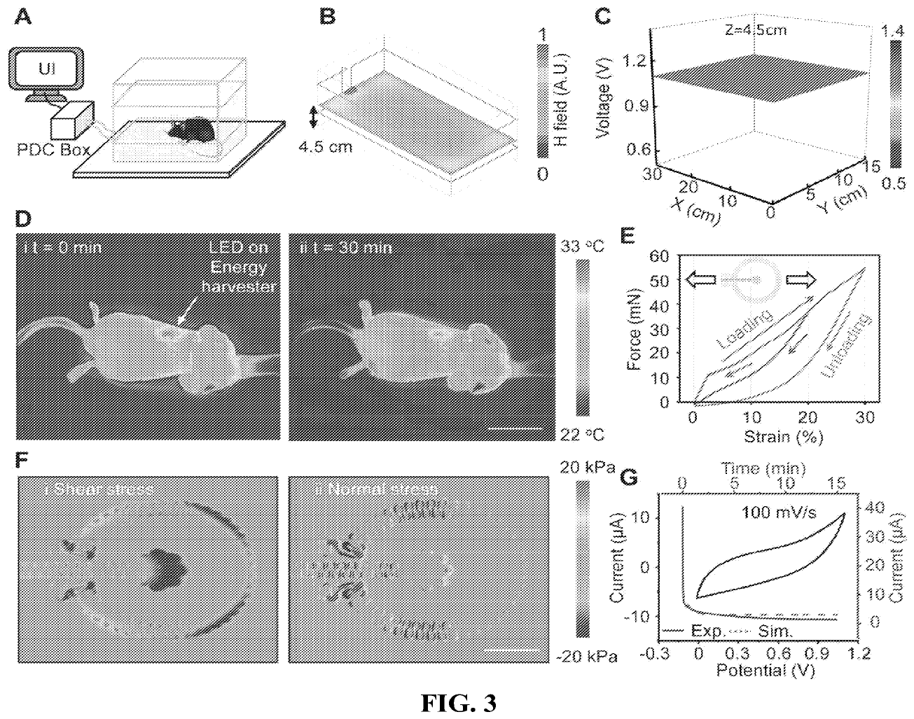

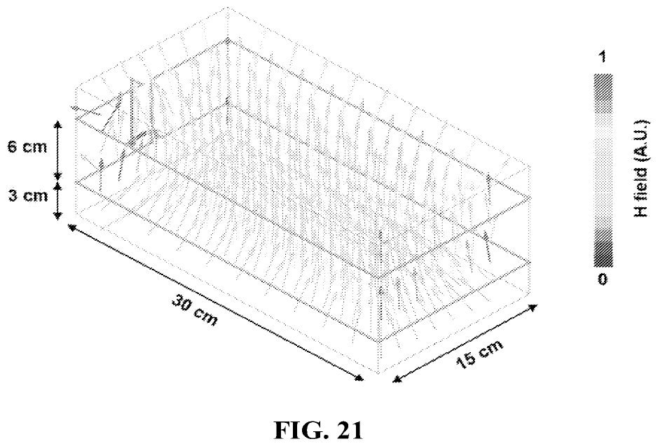

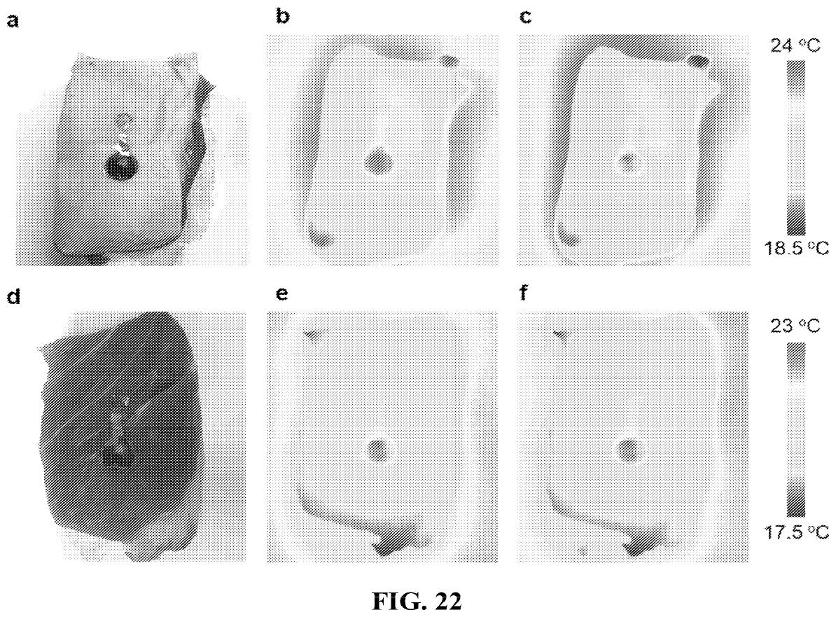

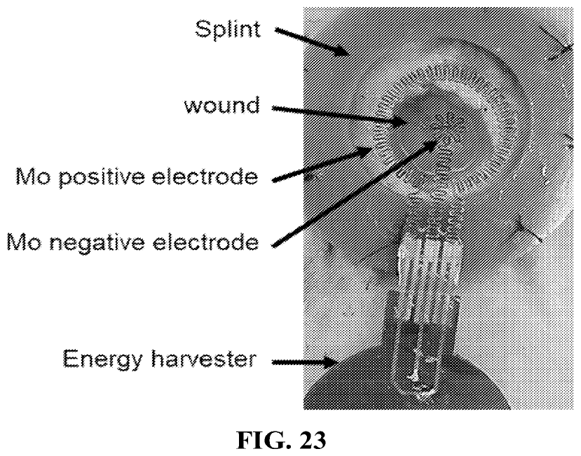

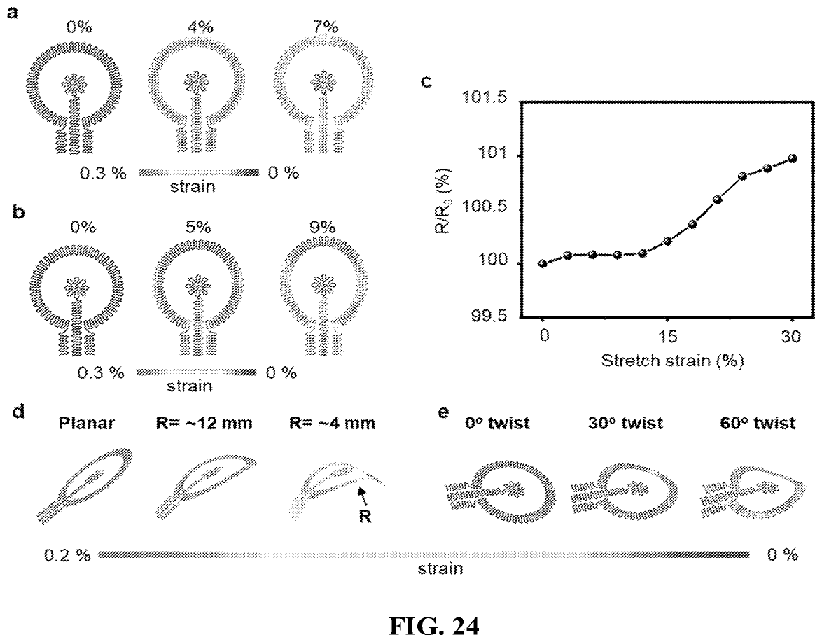

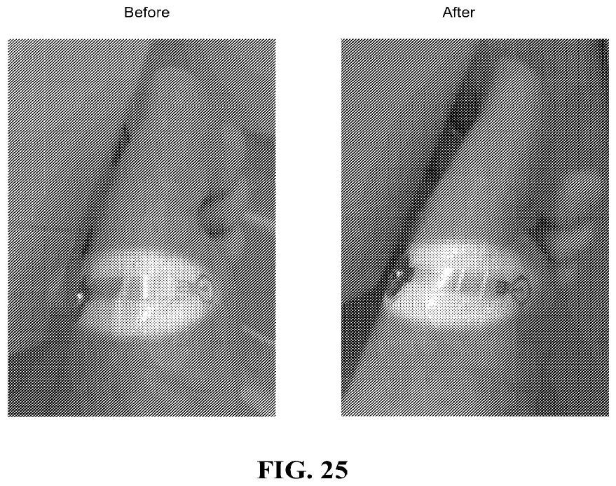

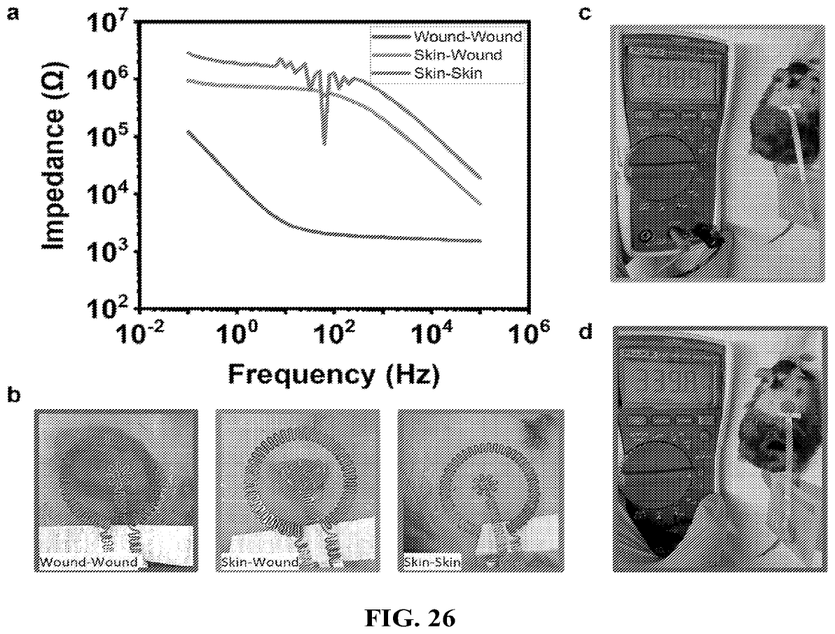

In vivo animal model studies use a cage with an inductive power transmitter coil wrapped around the structure to allow for wireless, battery-free operation without constraint on animal behaviors and movements. A graphical user interface operating on a separate computer supports real-time control over the stimulation parameters and serves as an interface to record the associated currents that pass through the anode and cathode. Simulations indicate that the transmitter coil establishes uniform electromagnetic fields at all regions except for the boundaries of the cage (panel B of FIG. 3 and FIG. 21). When the height of the BES device is 4.5 cm, comparable to the position of the back of a mouse, the output voltage is 1.1 V, as actively controlled by linear and low-dropout (LDO) regulators (panel C of FIG. 3). Infrared (IR) imaging indicates no significant heating associated with electrodes during stimulation (panel D of FIG. 3 and FIGS. 22-23). The surface of the wireless platform can reach temperatures of 33° C. while on the animal (body temperature of 27° C.). FEA simulations for the outer Mo electrode under uniaxial stretching show elastic behavior up to 9% stretching, corresponding to stretching of the skin in an isotropic manner to about 15% (panel E of FIG. 3 and FIG. 24). FEA simulations indicate that the shear and normal interfacial stresses in most areas remain below the threshold for sensation (about 20 kPa) for deformations of the skin to tensile strains of the Mo electrode up to about 9% (panel F of FIG. 3). Stretching to 20 and 30% leads to plastic deformation of the Mo by strains of 1 and 7%, respectively. The strain in the electrode structures is about 0.2% at a bending radius of 4 mm or a torsion angle of 60 degrees (FIG. 24). In clinical cases, patients wear a total contact cast to eliminate pressures on the foot ulcers. Experimental testing shows that the Mo electrode was also stable under continuous frictional forces and pressures associated with walking, without the cast (FIG. 25). The impedances between the electrodes when placed on the epidermis (case 1) and when the outer electrode is on the epidermis and the inner electrode is on the adipose tissue (case 2) are about 570 kΩ and about 200 kΩ at a frequency of 1 kHz, respectively. The impedance with both electrodes on adipose tissue (case 3) (FIG. 26) is about 100 times smaller than that of case 2. In vivo CV measurements with the Mo electrodes on the wound (case 2) across a voltage range from 0 to 1.1V shows that Mo electrodes do not undergo redox reactions when exposed to the epidermis, adipose tissue or biofluids (panel G of FIG. 3). Experimental results and simulations for the applied current between the inner and outer electrodes under a constant voltage of 1.1 V are consistent with a capacitive response, as expected based on ionic transport.

### Electrostimulation Accelerates Closure Rate and Granulation Tissue Formation in Diabetic Mice

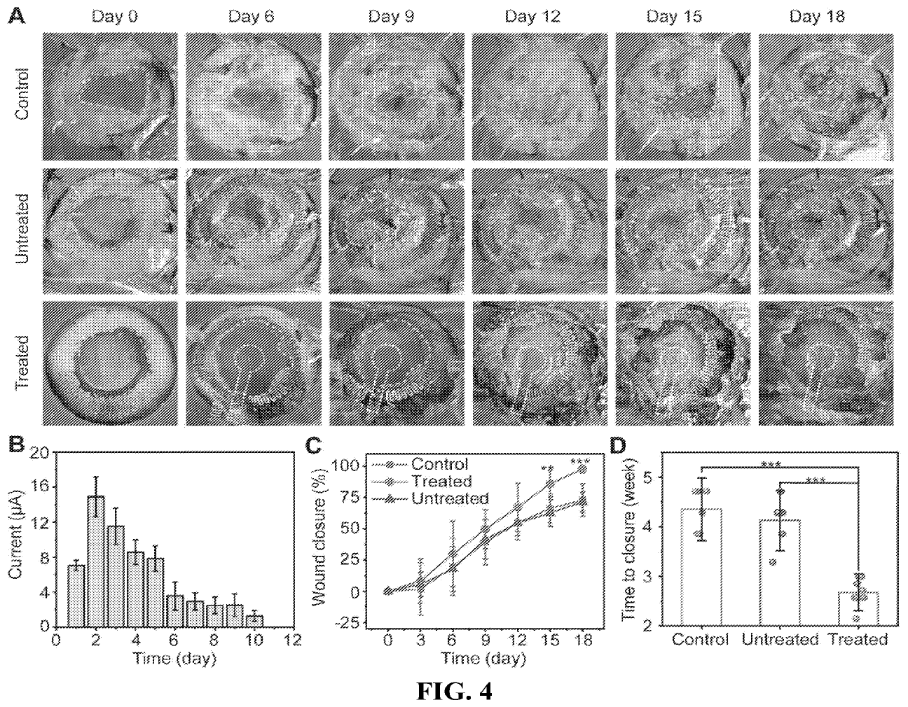

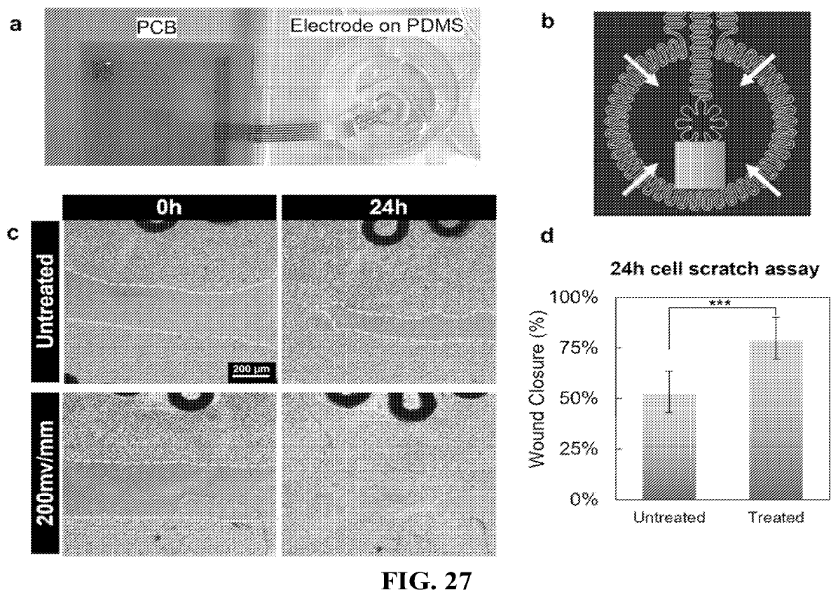

We hypothesized that accelerated in vitro keratinocyte migration toward the anode (FIG. 27) will translate to an enhanced wound healing response in vivo by re-introducing an endogenous electric field toward the center of the wound (FIG. 12). Evaluations of wound area changes and wound closures serve as the basis for comparing the healing progress between control and electrostimulated groups. The splinted, full thickness excisional dermal wound model in diabetic mice minimizes wound healing due to skin contraction and enables a healing process that includes granulation and re-epithelialization, similar to the healing processes for wounds in human skin. Panel A of FIG. 4 represents the healing progression of three different diabetic mouse groups, where the orange circle highlights the wound area (control, untreated, and treated mouse). The treated group involves Mo electrodes with continuous DC electrostimulation for 30 minutes every day until full wound closure; the untreated group involves Mo electrodes without electrostimulation; the control groups had no Mo electrode and no treatment other than a protective dressing. The electrodes must still be covered with a traditional dressing such as Tegaderm™ or similar to protect the wound site. The electrostimulation for 30 minutes per day until full wound closure was selected based on previous clinical studies. The studies focus on two circular excisional wounds (6 mm in diameter) on the left and right sides of the back for the treated/untreated groups and the control group, respectively. Digital pictures of the wound collected every three days reveal the extent of wound closure at these time points.

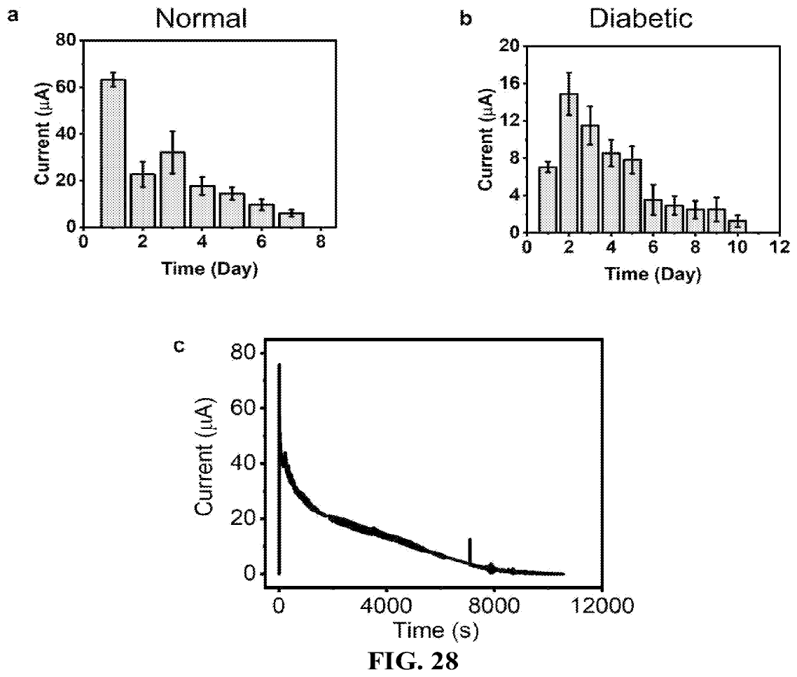

Wound exudate formed as part of the inflammatory response contains proteins, essential nutrients, etc., as a moist and electrically conductive environment on the wound. The amount of exudate decreases after the inflammatory and proliferation stages, and then the wound gradually dries. Reflecting these processes, after the full integration of the device into the surrounding, the current between the outside and inside Mo electrodes increases up to about 20 μA at the beginning of the inflammatory stage and gradually decreases to 0 when the wound is fully dry (panel B of FIG. 4). The system can detect up to the point when the wound gets fully dry. As dryness is a key factor in wound healing, our system can accurately monitor critical early-stage healing. Benchtop tests with a hydrogel confirm that drying of the wound affects the sensed current (FIG. 28). This drying causes the ion conductivity to decrease, leading to a reduction of the current to 0 μA. In this way, the current provides an estimate of the healing progress, as signature of which is drying of the wound. Experiments show that the current measured from normal mice decays faster (FIG. 28) than that of diabetic mice, consistent with the expected relative rates of healing.

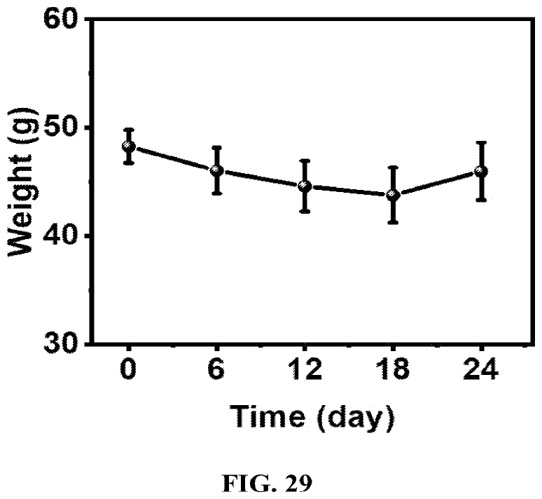

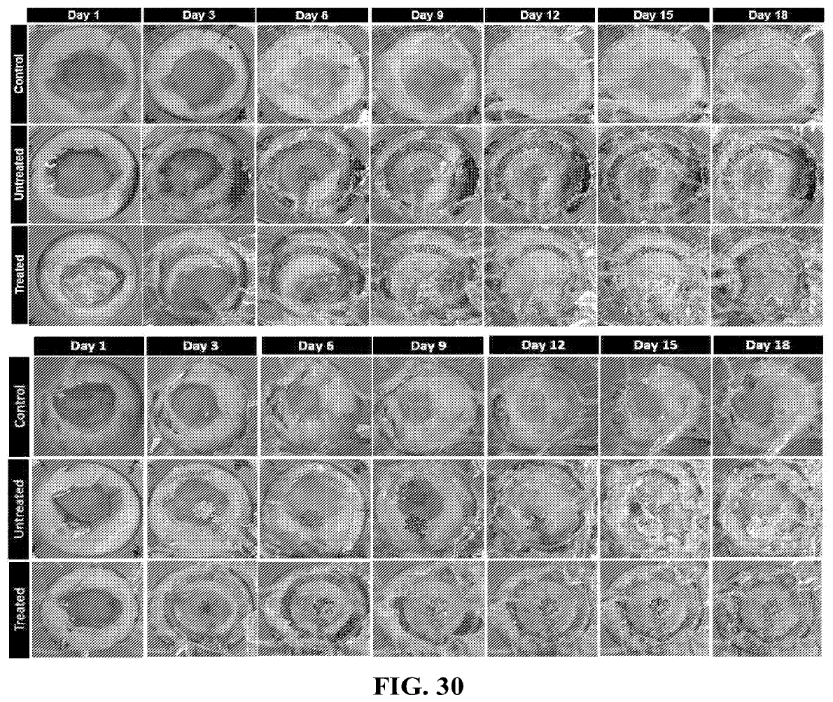

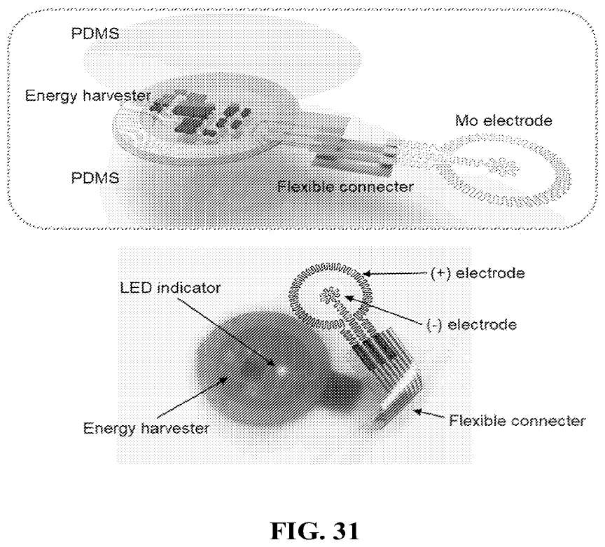

Electrostimulation using the devices and parameters reported here reduces the times for closure of excisional splinted wounds by about 30% compared to those of control and untreated groups. No significant weight changes occur during electrostimulation (FIG. 29). Also, the device itself does not change the wound healing rate, as determined by experiments performed without the electrostimulation treatment (9≤n≤11 per group, ** P<0.01, *** P<0.001) (panel C of FIG. 4 and FIG. 30). Wounds treated with electrostimulation heal at a significantly accelerated rate compared to other groups, such that 86.0+10% closure occurs on day 15 compared to 62.6 #11% for the untreated and 66.4+12% for the control groups. Most mice in the control (n=11) and untreated groups (n=5) require more than four weeks to complete wound closure. In comparison, closure occurs in the treated group (n=11) in less than three weeks (*** P<0.001, see panel D of FIG. 4). Additional experiments use a simplified BES with only the electrostimulation function (FIG. 31).

### Electrostimulation Promotes Re-Epithelialization and Angiogenesis in Wounds of Diabetic Animals

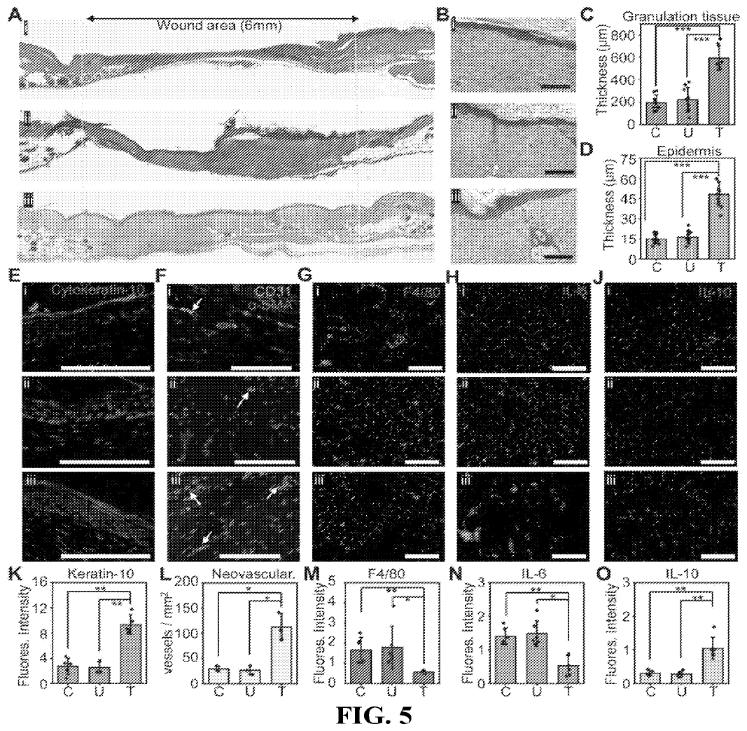

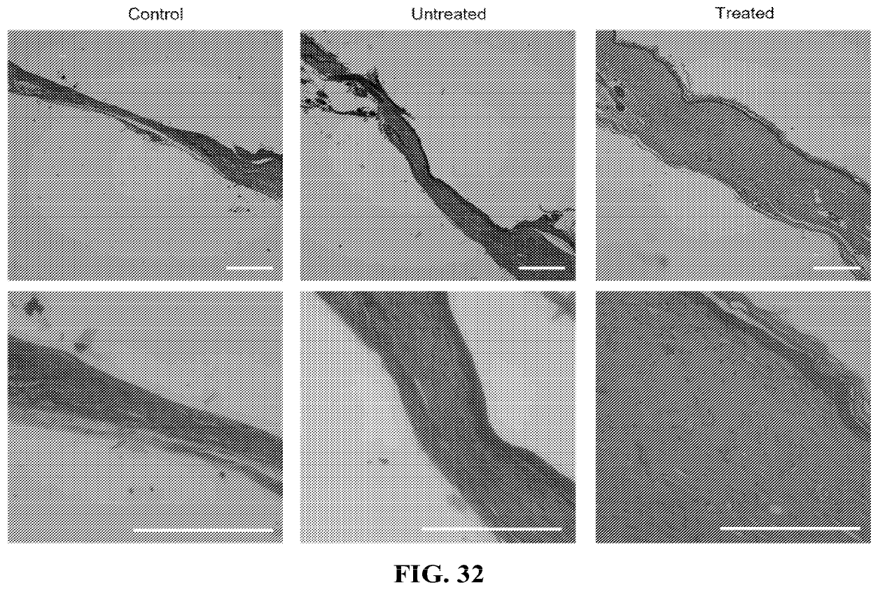

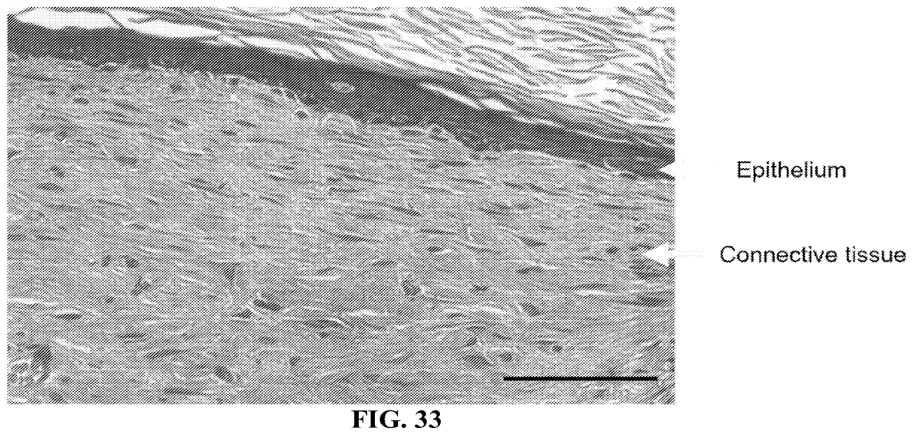

Histological studies quantify the granulation tissue thickness, epithelial thickness, and keratin-10 signal. The results of H&E staining of the wounded tissue on day 18 post-wounding appear in panel A of FIG. 5 and FIG. 32. The yellow dotted line encircles the wound site. The granulation tissue thickness for the control, untreated, and treated groups are 195±33 μm, 222±52 μm, and 595+50 μm, respectively (n=9, *** P<0.001 (panel C of FIG. 5). Masson's trichrome staining of the wound on day 30 post-wounding identifies epithelial coverage at the center of the wound (panel B of FIG. 5 and FIG. 33). As this is a validated, established impaired wound healing model, Masson's trichrome staining of the wound on day 30 post-wounding allows us to identify complete epithelial coverage at the center of the wound for all three groups. The density and the arrangement of collagen was similar in all three groups meaning that the thin electrode did not disturb the formation of the connective tissue. The epithelial thickness of the treated group (48±4 μm) is almost three times higher than that of the control (15±2 μm) and untreated (16±2 μm) groups (n=12, *** P<0.001) (panel D of FIG. 5). Results of keratin-10 signal and double staining of CD31 and α-SMA at day 30 post-wounding yield information on the maturation and differentiation of the outer and inner layer of the wound (panels E-F of FIG. 5). The electrostimulated group exhibits a matured spinous layer and strong keratin-10 signal. The keratin-10 fluorescence intensity for the control, untreated, and treated groups are 2.7±0.6, 2.5±0.4, and 9.3±0.7, respectively (n=5, ** P<0.01, see panel K of FIG. 5). This group also shows enhanced microvasculature formation (112±11 vessel/mm²) compared to the untreated (26±4 vessel/mm²) and control groups (29±2 vessel/mm²) (n=3, *P<0.05) (panel of L FIG. 5).

### Electrostimulation Subdues Inflammatory Responses

In chronic wounds, endogenous electric fields are typically absent due to prolonged inflammation (FIG. 12), thereby impairing the healing process. Electrostimulation is known to reduce pro-inflammatory and stimulate anti-inflammatory responses. Experiments to examine these effects involve three groups of the wounded tissue assessed via histology on day four post-wounding. Immunofluorescence staining for the macrophage cell marker, F4/80, and pro-inflammatory cytokine, IL-6, indicate that electrostimulation reduces inflammatory responses (panels G-H of FIG. 5). The mean fluorescence intensity associated with F4/80 is 1.6±0.3 for the control group, 1.8±0.5 for the untreated group, and 0.57±0.02 for the treated group (n=6) (panel M of FIG. 5). Similarly, fluorescence due to IL-6 is 1.4±0.1, 1.5±0.2, and 0.53±0.1 for the control, untreated, and treated groups, respectively (n=6) (panel N of FIG. 5). For the anti-inflammatory cytokine IL-10 (panel J of FIG. 5), the corresponding values are 0.30±0.03, 0.28 ±0.04 and 1.0±0.1 for the control, untreated and treated groups, respectively (n=6, *P<0.05, ** P<0.01) (panel O of FIG. 5). Remarkably, the electrodes do not induce any additional inflammatory responses compared to those of the control group. Electrostimulation thus leads to the transition from the early inflammation stage to the next stage by subduing the pro-inflammatory and stimulating the anti-inflammatory response.

### Mo Electrodes are Bioresorbable and Biocompatible In Vivo

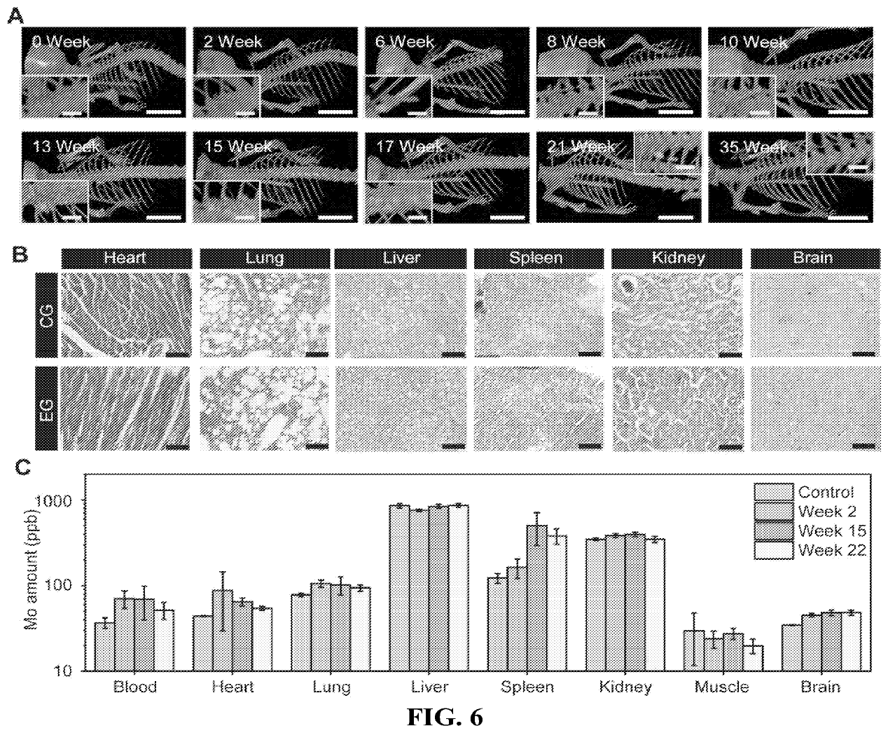

Micro-computed tomography (micro-CT) yields high-resolution images for monitoring the bioresorption of the Mo electrode. A small piece of the inner Mo electrode implanted into the mouse maintains its original shape over 13 weeks, meaning that the stimulation and sensing are consistent and predictable throughout the healing period, and then gradually resorbs in the body (panel A of FIG. 6). In vivo bioresorption tests demonstrate that the Mo electrode almost disappears after 35 weeks (245 days), which is similar to the estimated lifetime of the Mo electrode calculated by accelerated testing (around 300 days). Histology and biodistribution of Mo associated with bioresorption in a mouse model reveal aspects related to toxicity. Comparison between the control group (CG) and experimental group (EG) of the histological analysis of key organ tissues (heart, lung, liver, spleen, kidney, and brain; 22 weeks after Mo electrode implantation) indicates no damage to the tissue, no discernable immune response, and no distinguishable Mo flakes/particles (panel B of FIG. 6). Panel C of FIG. 6 shows the concentration of Mo in the blood, heart, lung, liver, spleen, kidney, muscle, and brain tissues explants from mice at 2, 15, and 22 weeks after implantation, measured by ICP-MS. The organs of the control group and those with implanted Mo electrodes reveal no abnormal accumulation of Mo in the organs, but a small accumulation during the first 2 weeks' implantation period. In the blood, heart, lung, and kidney, the Mo concentration gradually decreases and returns to a range similar to the control group after 22 weeks of implantation. Most of the Mo byproducts accumulate in the spleen, which easily stores nanoscale particles after weeks 15 of implantation. After 22 weeks, the micro-CT images confirm that almost all of the Mo resorbs in the body, and the concentration of Mo in the spleen also begins to decrease. In the brain, the concentration of Mo saturates after 22 weeks of implantation, and we expect them to be decreased at a later time point, as previously reported.

### DISCUSSION

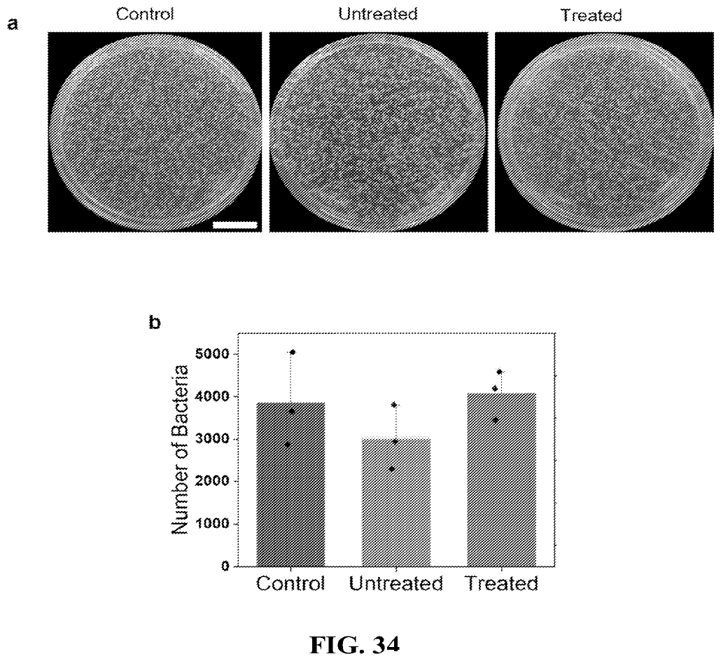

The results reported here demonstrate that a bioresorbable, wireless, and battery-free electrotherapy system can serve as an effective and unique platform for accelerating and monitoring the processes of wound healing in diabetic small animal models. The electrode designs support levels of electrical conductivity and interface impedances that are necessary for electrotherapy and wound monitoring over several weeks of use in a thin, flexible, and stretchable construct that naturally bioresorbs into the healed tissue to eliminate the need for surgical retrieval. The current measured during the stimulation serves as a parameter related to the healing process, dependent on drying of the wound as a crucial aspect of the healing process. The gradual decrease of current measurement relates directly to progressive healing. Pairing these compliant, bioresorbable electrodes with a compact wireless electronics module and graphical user interface yields a complete system with attributes that not only facilitate animal studies but also potentially treat and monitor chronic wounds in home settings. The placement of one device is expected to be sufficient to treat and monitor chronic wounds without the need for multiple skin care products. Although it is known that a high source of voltage inhibits bacterial growth, the naturally occurring endogenous current that we are attempting to restore or mimic did not have bactericidal effects (FIG. 34). However, we would expect the device to inhibit bacterial growth as Mo oxidizes into molybdenum oxides that have been reported to have bactericidal effects. This study is a first step in an established rodent model of diabetic impaired wound healing that assesses re-epithelialization and new tissue formation due to the presence of the splints that minimize wound contraction. Although this limitation requires caution when interpreting potential results in humans, the rodent model will enable us to investigate and develop additional enhancements such as integrating capabilities for programmed drug delivery, biochemical/biophysical sensing, and closed-loop control of operational parameters, which must also be evaluated in a larger animal model.

The foregoing description of the exemplary embodiments of the invention has been presented only for the purposes of illustration and description and is not intended to be exhaustive or to limit the invention to the precise forms disclosed. Many modifications and variations are possible in light of the above teaching.

The embodiments were chosen and described in order to explain the principles of the invention and their practical application so as to enable others skilled in the art to utilize the invention and various embodiments and with various modifications as are suited to the particular use contemplated. Alternative embodiments will become apparent to those skilled in the art to which the invention pertains without departing from its spirit and scope. Accordingly, the scope of the invention is defined by the appended claims rather than the foregoing description and the exemplary embodiments described therein.

Some references, which may include patents, patent applications and various publications, are cited and discussed in the description of this invention. The citation and/or discussion of such references is provided merely to clarify the description of the invention and is not an admission that any such reference is “prior art” to the invention described herein. All references cited and discussed in this specification are incorporated herein by reference in their entireties and to the same extent as if each reference was individually incorporated by reference.

### LIST OF REFERENCES

[1]. Economic Costs of Diabetes in the US in 2017. Diabetes Care, 41, 917 (2018).
[2]. L. Yazdanpanah, H. Shahbazian, I. Nazari, H. R. Arti, F. Ahmadi, S. E. Mohammadianinejad, B. Cheraghian, S. Hesam, Incidence and risk factors of diabetic foot ulcer: a population-based diabetic foot cohort (ADFC study)-two-year follow-up study.” International journal of endocrinology 2018 (2018).
[3]. P. K. Moulik, R. Mtonga, G. V. Gill, Amputation and mortality in new-onset diabetic foot ulcers stratified by etiology. Diabetes care, 26, 491-494 (2003).
[4]. G. FrykbergRobert, Challenges in the treatment of chronic wounds. Advances in wound care (2015).
[5]. S. A. Guo, L. A. DiPietro, Factors affecting wound healing. Journal of dental research, 89, 219-229 (2010).
[6]. M. M. Martino, P. S. Briquez, E. Güç, F. Tortelli, W. W. Kilarski, S. Metzger, J. J. Rice, G. A. Kuhn, R. Müller, M. A. Swartz, J. A. Hubbell, Growth factors engineered for super-affinity to the extracellular matrix enhance tissue healing. Science, 343, 885-888 (2014).
[7]. D. R. Griffin, M. M. Archang, C. H. Kuan, W. M. Weaver, J. S. Weinstein, A. C. Feng, A. Ruccia, E. Sideris, V. Ragkousis, J. Koh, M. V. Plikus, Activating an adaptive immune response from a hydrogel scaffold imparts regenerative wound healing. Nature materials, 20, 560-569 (2021).
[8]. A. Nourian Dehkordi, F. Mirahmadi Babaheydari, M. Chehelgerdi, S. Raeisi Dehkordi, Skin tissue engineering: wound healing based on stem-cell-based therapeutic strategies. Stem cell research & therapy, 10, 1-20 (2019).
[9]. K. Vig, A. Chaudhari, S. Tripathi, S. Dixit, R. Sahu, S. Pillai, V. A. Dennis, S. R. Singh, Advances in skin regeneration using tissue engineering. International journal of molecular sciences, 18, 789 (2017).
[10]. S. Gregor, M. Maegele, S. Sauerland, J. F. Krahn, F. Peinemann, S. Lange, Negative pressure wound therapy: a vacuum of evidence?. Archives of surgery, 143, 189-196 (2008).
[11]. G. Thakral, J. LaFontaine, B. Najafi, T. K. Talal, P. Kim, L. A. Lavery, Electrical stimulation to accelerate wound healing. Diabetic foot & ankle, 4, 22081 (2013).
[12]. H. Kai, T. Yamauchi, Y. Ogawa, A. Tsubota, T. Magome, T. Miyake, K. Yamasaki, N. Nishizawa, 2017. Accelerated wound healing on skin by electrical stimulation with a bioelectric plaster. Advanced healthcare materials, 6, 1700465 (2017)
[13]. C. Lim, Y. J. Hong, J. Jung, Y. Shin, S. H. Sunwoo, S. Baik, O. K. Park, S. H. Choi, T. Hyeon, J. H. Kim, S. Lee, Tissue-like skin-device interface for wearable bioelectronics by using ultrasoft, mass-permeable, and low-impedance hydrogels. Science Advances, 7, eabd3716 (2021).
[14]. D. W. Park, J. P. Ness, S. K. Brodnick, C. Esquibel, J. Novello, F. Atry, D. H. Baek, H. Kim, J. Bong, K. I. Swanson, A. J. Suminski, Electrical neural stimulation and simultaneous in vivo monitoring with transparent graphene electrode arrays implanted in GCaMP6f mice. ACS nano, 12, 148-157 (2018) . . .
[15]. M. R. MacEwan, E. R. Zellmer, J. J. Wheeler, H. Burton, D. W. Moran, Regenerated sciatic nerve axons stimulated through a chronically implanted macro-sieve electrode. Frontiers in neuroscience, 10, 557 (2016).
[16]. B. Reid, M. Zhao, The electrical response to injury: molecular mechanisms and wound healing. Advances in wound care, 3, 184-201 (2014).
[17]. M. Zhao, B. Song, J. Pu, T. Wada, B. Reid, G. Tai, F. Wang, A. Guo, P. Walczysko, Y. Gu, T. Sasaki, Electrical signals control wound healing through phosphatidylinositol-3-OH kinase-y and PTEN. Nature, 442, 457-460 (2006).
[18]. T. B. Saw, X. Gao, M. Li, J. He, A. P. Le, S. Marsh, K. H. Lin, A. Ludwig, J. Prost, C. T. Lim, Transepithelial potential difference governs epithelial homeostasis by electromechanics. Nature Physics, 1-7 (2022).
[19]. S. K. Kang, S. W. Hwang, S. Yu, J. H. Seo, E. A. Corbin, J. Shin, D. S. Wie, R. Bashir, Z. Ma, J. A. Rogers, Biodegradable thin metal foils and spin-on glass materials for transient electronics. Advanced Functional Materials, 25, 1789-1797 (2015).
[20]. G. Lee, S. K. Kang, S. M. Won, P. Gutruf, Y. R. Jeong, J. Koo, S. S. Lee, J. A. Rogers, J. S. Ha, Fully biodegradable microsupercapacitor for power storage in transient electronics. Advanced Energy Materials, 7, 1700157 (2017).
[21]. G. Lee, E. Ray, H. J. Yoon, S. Genovese, Y. S. Choi, M. K. Lee, S. Şahin, Y. Yan, H. Y. Ahn, A. J. Bandodkar, J. Kim, M. Park, H. Ryu, S. S. Kwak, Y. H. Jung, A. Odabas, U. Khandpur, W. Z. Ray, M. R. Macewan, J. A. Rogers, A bioresorbable peripheral nerve stimulator for electronic pain block. Science Advances, 8, 9169 (2022)
[22]. Y. S. Choi, H. Jeong, R. T. Yin, R. Avila, A. Pfenniger, J. Yoo, J. Y. Lee, A. Tzavelis, Y. J. Lee, S. W. Chen, H. S. Knight, S. Kim, H. Y. Ahn, G. Wickerson, A. Vázquez-Guardado, E. Higbee-Dempsey, B. A. Russo, M. A. Napolitano, T. J. Holleran, L. A. Razzak, A. N. Miniovich, G. Lee, B. Geist, B. Kim, S. Han, J. A. Brennan, K. Aras, S. S. Kwak, J. Kim, E. A. Waters, X. Yang, A. Burrell, K. S. Chun, C. Liu, C. Wu, A. Y. Rwei, A. N. Spann, A. Banks, D. Johnson, Z. J. Zhang, C. R. Haney, S. H. Jin, A. V. Sahakian, Y. Huang, G. D. Trachiotis, B. P. Knight, R. K. Arora, I. R. Efimov, J. A. Rogers, A transient, closed-loop network of wireless, body-integrated devices for autonomous electrotherapy. Science, 376, 1006-1012 (2022).
[23]. G. S. Frankel, Pitting corrosion of metals: a review of the critical factors. Journal of the Electrochemical society, 145, 2186 (1998).
[24]. W. D. Callister, D. G. Rethwisch, Materials science and engineering: an introduction (Vol. 9). New York: Wiley (2018).
[25]. Y. Liu, M. Pharr, G. A. Salvatore, Lab-on-skin: a review of flexible and stretchable electronics for wearable health monitoring. ACS nano, 11, 9614-9635 (2017).
[26]. A. Kaneko, N. Asai, T. Kanda, The influence of age on pressure perception of static and moving two-point discrimination in normal subjects. Journal of hand therapy, 18, 421-425 (2005).
[27]. P. Sheehan, P. Jones, A. Caselli, J. M. Giurini, A. Veves, Percent change in wound area of diabetic foot ulcers over a 4-week period is a robust predictor of complete healing in a 12-week prospective trial. Diabetes care, 26, 1879-1882 (2003).
[28]. R. D. Galiano, V. J. Michaels, M. Dobryansky, J. P. Levine, G. C. Gurtner, Quantitative and reproducible murine model of excisional wound healing. Wound repair and regeneration, 12, 485-492 (2004).
[29]. J. S. Petrofsky, D. Lawson, L. Berk, H. Suh, Enhanced healing of diabetic foot ulcers using local heat and electrical stimulation for 30 min three times per week. Journal of diabetes, 2, 41-46 (2010).
[30]. D. Lawson, J. S. Petrofsky, A randomized control study on the effect of biphasic electrical stimulation in a warm room on skin blood flow and healing rates in chronic wounds of patients with and without diabetes. Medical science monitor, 13, 258-263 (2007).
[31]. J. A. Feedar, L. C. Kloth, G. D. Gentzkow, Chronic dermal ulcer healing enhanced with monophasic pulsed electrical stimulation. Physical Therapy, 71, 639-649 (1991).
[32]. E. M. Tottoli, R. Dorati, I. Genta, E. Chiesa, S. Pisani, B. Conti, Skin wound healing process and new emerging technologies for skin wound care and regeneration. Pharmaceutics, 12, 735 (2020).
[33]. G. Han, R. Ceilley, Chronic wound healing: a review of current management and treatments. Advances in therapy, 34, 599-610 (2017).
[34]. J. Michaels, S. S. Churgin, K. M. Blechman, M. R. Greives, S. Aarabi, R. D. Galiano, G. C. Gurtner, db/db mice exhibit severe wound-healing impairments compared with other murine diabetic strains in a silicone-splinted excisional wound model. Wound repair and regeneration, 15, 665-670 (2007).
[35]. Sebastian, A., Syed, F., Perry, D., Balamurugan, V., Colthurst, J., Chaudhry, I. H., & Bayat, A. Acceleration of cutaneous healing by electrical stimulation: Degenerate electrical waveform down-regulates inflammation, up-regulates angiogenesis and advances remodeling in temporal punch biopsies in a human volunteer study. Wound Repair and Regeneration, 19, 693-708 (2011).
[36]. S. G. Gürgen, O. Sayn, F. çetin, A. Tuç Yücel, Transcutaneous electrical nerve stimulation (TENS) accelerates cutaneous wound healing and inhibits pro-inflammatory cytokines. Inflammation, 37, 775-784 (2014).
[37]. M. Cataldi, C. Vigliotti, T. Mosca, M. Cammarota, D. Capone, Emerging role of the spleen in the pharmacokinetics of monoclonal antibodies, nanoparticles and exosomes. International journal of molecular sciences, 18, 1249 (2017).
[38]. A. Schauer, C. Redlich, J. Scheibler, G. Poehle, P. Barthel, A. Maennel, V. Adams, T. Weissgaerber, A. Linke, P. Quadbeck, Biocompatibility and Degradation Behavior of Molybdenum in an In Vivo Rat Model. Materials, 14, 7776 (2021).
[39]. S. B. Rajendran, K. Challen, K. L. Wright, J. G. Hardy, Electrical stimulation to enhance wound healing. Journal of Functional Biomaterials, 12, 40 (2021).
[40]. G. Duan, L. Wen, X. Sun, Z. Wei, R. Duan, J. Zeng, J. Cui, C. Liu, Z. Yu, X. Xie, M. Gao, Healing Diabetic Ulcers with Mo03-X Nanodots Possessing Intrinsic ROS-Scavenging and Bacteria-Killing Capacities. Small, 18, 2107137 (2022).
[41]. COMSOL Multiphysics. Material Library. Manual 66 (2012).
[42]. J. C. Wei, G. A. Edwards, D. J. Martin, H. Huang, M. L. Crichton, M. A. Kendall, Allometric scaling of skin thickness, elasticity, viscoelasticity to mass for micro-medical device translation: From mice, rats, rabbits, pigs to humans. Sci. Rep. 7, 1-17 (2017).
[43]. M. Tonghui, M. Hara, R. Sougrat, J. M. Verbavatz, A. S. Verkman, Impaired stratum corneum hydration in mice lacking epidermal water channel aquaporin-3. J. Biol. Chem. 277, 17147-17153 (2002).
[44]. S. L. Jacques, S. A. Prahl, Modeling optical and thermal distributions in tissue during laser irradiation. Lasers Surg. Med. 6, 494-503 (1987).
[45]. M. L. Cohen, Measurement of the thermal properties of human skin. A review. J. Invest. Dermatol. 69, 333-338 (1977).
[46]. C. Gabriel, S. Gabriel, E. Corthout, The dielectric properties of biological tissues: I. Literature survey. Phys. Med. Biol. 41, 2231-2249 (1996).
[47]. S. Gabriel, R. W. Lau, C. Gabriel, The dielectric properties of biological tissues: II. Measurements in the frequency range 10 Hz to 20 GHz. Phys. Med. Biol. 41, 2251-2269 (1996).
[48]. L. S. Hansen, J. E. Coggle, J. Wells, M. W. Charles, The influence of the hair cycle on the thickness of mouse skin. Anat. Rec. 210, 569-573 (1984).
[49]. O. A. Bahri, N. Naldaiz-Gastesi, D. C. Kennedy, A. M. Wheatley, A. Izeta, K. J. McCullagh, The panniculus carnosus muscle: A novel model of striated muscle regeneration that exhibits sex differences in the mdx mouse. Sci. Rep. 9, 1-15 (2019).
[50]. M. S. Farvid, T. W. K. Ng, D. C. Chan, P. H. R. Barrett, G. F. Watts, Association of adiponectin and resistin with adipose tissue compartments, insulin resistance and dyslipidemia. Diabetes, Obes. Metab. 7, 406-413 (2005).
[51]. P. Faber, L. Garby, Fat content affects heat capacity: a study in mice. Acta Physiol. Scand. 153, 185-187 (1995).
[52]. K. Crawford, R. Flick, L. Close, D. Shelly, R. Paul, K. Bove, A. Kumar, J. Lessard, Mice Lacking Skeletal Muscle Actin Show Reduced Muscle Strength and Growth Deficits and Die during the Neonatal Period. Mol. Cell. Biol. 22, 5887-5896 (2002).
[53]. A. Bonetto, D. C. Andersson, D. L. Waning, Assessment of muscle mass and strength in mice. Bonekey Rep. 4, 1-10 (2015).
[54]. A. W. Stewart, Myothermic Calibration for Skeletal Muscle. Basic Appl. Myol. 1, 157-161 (1991).
[55]. J. W. Valvano, J. T. Allen, H. F. Bowman, Simultaneous Measurement of Thermal Conductivity, Thermal Diffusivity, and Perfusion in Small Volumes of Tissue. Am. Soc. Mech. Eng. (1981).

1. An electrotherapy system, comprising:
a pair of electrodes coupled with a region of interest of a subject for providing electrostimulation thereto; and
a wireless platform coupled with the pair of electrodes for operable providing power to the stimulator.

2. The electrotherapy system of claim 1, wherein the pair of electrodes has a first electrode attached onto the region of interest and a second electrode surrounding the first electrode.

3. The electrotherapy system of claim 2, wherein the pair of electrodes is spatially apart from each other to define an electrode spacing that is larger than 1 mm.

4. The electrotherapy system of claim 2, wherein the first electrode is an inner electrode placed at the center of the region of interest and the second electrode is an outer electrode placed slightly outside of the region of interest around its perimeter.

5. The electrotherapy system of claim 4, wherein the first electrode and the second electrode are concentrically arranged such that the pair of electrodes is a concentric pair of electrodes.

6. The electrotherapy system of claim 2, wherein each of the pair of electrodes is formed with a filamentary serpentine layout so that each electrode is mechanical flexible and stretchable.

7. The electrotherapy system of claim 6, wherein each electrode has a thickness in a range of 10-30 μm and a width in range of 50-200 μm.

8. The electrotherapy system of claim 7, wherein the first electrode includes a serpentine trace with a thickness of 15 μm and width of 120 μm in a flower-like design;
and the second electrode adopts a similar serpentine shape with similar thickness and width.

9. The electrotherapy system of claim 2, wherein the pair of electrodes is bioresorbable and biocompatible.

10. The electrotherapy system of claim 2, wherein the pair of electrodes is formed of a bioresorbable conductive material comprising molybdenum (Mo), zinc (Zn), iron (Fe), tungsten (W), magnesium (Mg), AZ31B (3 wt % Al and 1 wt % Zn) Mg alloy, and/or other bioresorbable conductive materials.

11. The electrotherapy system of claim 2, wherein the pair of electrodes is formed without an encapsulation layer.

12. The electrotherapy system of claim 2, wherein the wireless platform comprises:
a power harvesting unit that operably powers the system;
a near field communication (NFC) system on chip (SoC) that operably supports wireless communication; and
a microcontroller unit (MCU) that operably supplies a voltage to the electrodes for stimulation and measures current between the electrodes.

13. The electrotherapy system of claim 12, wherein the wireless platform further comprises a component that serves as an audio or visual indicator of system operation.

14. The electrotherapy system of claim 13, wherein the visual indicator of system operation is a light-emitting diode (LED).

15. The electrotherapy system of claim 12, wherein the wireless power harvesting unit comprises an antenna for delivering the power to the system.

16. The electrotherapy system of claim 15, wherein the power harvesting unit operates by inductive coupling at a resonance frequency in a range of about 10-15 Mhz, preferably about 13.56 MHz.

17. The electrotherapy system of claim 1, further comprising an encapsulation structure encapsulating the wireless platform.

18. The electrotherapy system of claim 17, wherein the encapsulation structure is formed of a silicone elastomer, polymer, and/or dielectric materials.

19. The electrotherapy system of claim 12, wherein the region of interest is a wound site of the subject, and wherein the electrotherapy system is adapted for electrotherapeutically treating the wound and monitoring the processes of wound healing.

20. The electrotherapy system of claim 19, wherein the pair of electrodes is designed to support levels of electrical conductivity and interface impedances that are necessary for electrotherapy and wound monitoring over several weeks of use in a thin, flexible, and stretchable construct that naturally bioresorbs into the healed tissue to eliminate the need for surgical retrieval.

21. The electrotherapy system of claim 20, wherein the inner electrode is placed on adipose tissue of the wound and the outer electrode is placed on the epidermis to mimic or reproduce in vivo conditions.

22. The electrotherapy system of claim 21, wherein the inner electrode is fixed on the wound site by a sutured splint ring structure.

23. The electrotherapy system of claim 22, wherein the inner electrode is eliminable completely from the wound site of the subject through natural chemical/biochemical processes of hydrolysis and/or metabolic actions, over a subsequent timeframe following completion of electrotherapy.

24. The electrotherapy system of claim 21, wherein the voltage is applied to the electrodes for electrostimulation for predetermined periods of time every day until full wound closure.

25. The electrotherapy system of claim 24, wherein the predetermined periods of time is customizable.

26. The electrotherapy system of claim 24, wherein the current measured during the stimulation is associated with a dying process of the wound and provides an estimate of the healing progress, as a signature of which is drying of the wound.

27. The electrotherapy system of claim 26, wherein a gradual decrease of the current relates directly to progressive healing of the wound, and the currently gradually decreases to 0 when the wound is fully dry.

28. The electrotherapy system of claim 19, wherein the electrostimulation results in an inward direct current from the healthy site to the wounded area that mimics naturally driven endogenous wound currents.

29. The electrotherapy system of claim 19, wherein the electrostimulation results in acceleration of closure rate and granulation tissue formation of the wound; and/or
promotion of re-epithelialization and angiogenesis in the wound.

30. The electrotherapy system of claim 29, wherein the electrostimulation reduces the times for closure of excisional splinted wounds by about 30% or more, compared to those of control and untreated groups.

31. The electrotherapy system of claim 19, wherein the electrostimulation results in a transition from an early inflammation stage to the next stage by subduing the pro-inflammatory and stimulating the anti-inflammatory response.

32. The electrotherapy system of claim 1, further comprising a releasable flexible connector electrically connected between the stimulator and the wireless platform.

33. The electrotherapy system of claim 32, wherein the flexible connector is configured to allow the wireless platform to be positioned onto healthy skin nearby the wound site.

34. The electrotherapy system of claim 1, wherein the stimulator and the wireless platform are directly connected to each other.

35. The electrotherapy system of claim 1, further comprising a customized app with a graphical user interface operating on an external device, configured to support real-time control over stimulation parameters, serve as an interface to record the current for monitoring the progress of wound healing.

36. The electrotherapy system of claim 35, wherein the external device is a mobile device, a computer, or a cloud service.

37. The electrotherapy system of claim 1, further comprising:
means for drug delivery, biochemical/biophysical sensing, and/or closed-loop control of operational parameters.

38. The electrotherapy system of claim 1, wherein the electrotherapy system is a bioresorbable, wireless, and battery-free electrotherapy system.

39. An electrotherapy system, comprising:
a stimulator coupled with a region of interest of a subject for providing electrostimulation thereto.

40. The electrotherapy system of claim 39, wherein the stimulator comprises a pair of electrodes having a first electrode attached to the region of interest and a second electrode surrounding the first electrode.

41. The electrotherapy system of claim 40, wherein the pair of electrodes is bioresporbable and biocompatible.

42. The electrotherapy system of claim 40, further comprising:
a microcontroller unit configured to supply a voltage to the electrodes and measure current between the electrodes.

43. The electrotherapy system of claim 42, further comprising:
a wireless power harvesting unit configured to deliver power via resonant inductive coupling to the microcontroller unit; and
a near field communication (NFC) system on chip (SoC) that operably supports wireless communication.

44. The electrotherapy system of claim 42, wherein the voltage is applied to the electrodes such that electrostimulation results in an inward direct current from the healthy site to the wounded area that mimics naturally driven endogenous wound currents.

45. The electrotherapy system of claim 42, further comprising a customized app with a graphical user interface operating on an external device, configured to support real-time control over stimulation parameters, serve as an interface to record the current for monitoring the progress of wound healing.

46. A method for electrotherapeutic stimulation to a region of interest of a subject, comprising:
providing a pair of electrodes having an inner electrode attached onto the region of interest and an outer electrode surrounding the inner electrode; and
applying a voltage to the pair of electrodes for electrostimulation to the region of interest for predetermined periods of time every day until full wound closure,
wherein the region of interest is a wound site of the subject.

47. The method of claim 46, wherein said applying the voltage to the pair of electrodes comprises:
wirelessly transmitting power to a microcontroller unit by a wireless power harvesting unit via resonant inductive coupling; and
applying the voltage from the microcontroller unit to the pair of electrodes that is electrically connected to the microcontroller unit.

48. The method of claim 47, wherein the power harvesting unit operates by resonant inductive coupling at a resonance frequency in a range of about 10-15 Mhz, preferably about 13.56 MHz.

49. The method of claim 46, wherein the electrostimulation results in an inward direct current from the healthy site to the wounded area that mimics naturally driven endogenous wound currents.

50. The method of claim 46, further comprising:
measuring current between the inner electrode and the outer electrode; and
estimating the healing progress of the wound from the current measured during the stimulation,
wherein the current is associated with a dying process of the wound.

51. The method of claim 50, wherein a gradual decrease of the current relates directly to progressive healing of the wound, and the currently gradually decreases to 0 when the wound is fully dry.

52. The method of claim 46, wherein the electrostimulation results in an electric field strength of about 1 mV/mm or more near the inner electrode and in regions of adipose tissue between the outer and inner electrodes, which is sufficient to cause migration of human keratinocyte cells to accelerate wound healing processes.

53. The method of claim 46, wherein the electrostimulation results in acceleration of closure rate and granulation tissue formation of the wound; and/or promotion of re-epithelialization and angiogenesis in the wound.

54. The method of claim 53, wherein the electrostimulation reduces the times for closure of excisional splinted wounds by about 30% or more, compared to those of control and untreated groups.

55. The method of claim 46, wherein the electrostimulation results in a transition from an early inflammation stage to the next stage by subduing the pro-inflammatory and stimulating the anti-inflammatory response.

56. The method of claim 46, wherein the inner electrode is eliminable completely from the wound site of the subject through natural chemical/biochemical processes of hydrolysis and/or metabolic actions, over a subsequent timeframe following completion of electrotherapy.

57. The method of claim 46, further comprising:
delivering one or more drugs to the wound site;
detecting biochemical/biophysical parameters associated with the wound site; and/or
performing closed-loop control of operational parameters.
